(19) United States
(12) Patent Application Publication
(10) Pub. No.: US 2019/0185945 A1
(43) Pub. Date: Jun. 20, 2019

(54) Biomarkers of Oral, Pharyngeal and Laryngeal Cancers

(71) Applicant: UNIVERSITY OF TECHNOLOGY SYDNEY, New South Wales (AU)

(72) Inventors: Nham Tran, Casula (AU); Samantha Khoury, Constitution Hill (AU)

(21) Appl. No.: 16/326,140

(22) PCT Filed: Aug. 18, 2017
(86) PCT No.: PCT/AU2017/050887

§ 371 Date: Feb. 15, 2019

(30) Foreign Application Priority Data
Aug. 18, 2016 (AU) 2016903272

(51) Int. Cl. C12Q 1/6886

(52) U.S. Cl. CPC C12Q 1/6886; C12Q 2600/178; C12Q 2600/106; C12Q 2600/112; C12Q 2600/158

(57) ABSTRACT

Provided herein are methods for detecting a head and neck cancer of the oral cavity or throat, optionally oral squamous cell carcinoma, comprising executing the step of determining the expression of two or more miRNA in a biological sample obtained from a subject, wherein the two or more miRNA are selected from the group consisting of hsa-let-7a, hsa-miR-16, hsa-miR-21, hsa-miR-451, hsa-miR-486-5p and hsa-miR-92a-3p, and wherein the level of expression of said two or more miRNAs in the biological sample relative to the level of expression of said two or more miRNAs in one or more cancer-free reference samples is indicative of the presence of a head and neck cancer of the oral cavity or throat of the subject.

### TECHNICAL FIELD

The present disclosure relates generally to methods and protocols for the diagnosis and prognosis of head and neck cancers of the oral cavity or throat, in particular oral, pharyngeal and laryngeal cancers, and more particularly oral, oropharyngeal, pharyngeal and laryngeal squamous cell carcinomas.

### BACKGROUND OF THE DISCLOSURE

Despite advances in our understanding and therapeutic treatment of many forms of cancer, cancer remains one of leading causes of death around the world, with prevalence of many cancers on the increase. Oral, pharyngeal and laryngeal cancers affect organs critical to basic functions such as speech and swallowing and typically have a large impact on quality of life. They also typically have a poor prognosis and are associated with significant morbidity, approximately 43% of sufferers not surviving beyond five years from diagnosis.

Rates of oral cancer are increasing year on year. In the United States alone approximately 42,000 people are diagnosed with oral cancer each year and it is the most common cancer in India with over 50,000 cases diagnosed annually. Concerningly, the age of diagnosis is also lowering. Whilst oral cancer remains most prevalent in males, due largely to higher rates of smoking and alcohol consumption, females between 40-49 years of age are the fast growing group of sufferers diagnosed. Also of significant public health concern is the rise of human papilloma virus 16 (HPV16) infections in the oropharynx, tonsils, and at the base of the tongue. HPV16 is a major cause of oropharyngeal squamous cell carcinoma in the United States and the proportion of oropharyngeal cancers associated with HPV16 is growing around the world.

Oral, pharyngeal and laryngeal cancers often produce few symptoms until well advanced. As a result, a significant proportion of patients first present with advanced (Stage 3 or 4) disease, and this is the principal cause of the high rate of morbidity. Prognosis can be improved significantly with early and effective diagnosis. For example with early detection, survival rates of oral cancer sufferers can improve dramatically to 80-90%. Early diagnosis allows early intervention with the most effective therapeutic treatments and/or patient management. However at present diagnosis of oral, pharyngeal and laryngeal cancers typically requires an invasive and painful tumour biopsy, such as fine needle aspiration. There are no clinically available biomarkers enabling the early detection of these cancers.

### SUMMARY OF THE DISCLOSURE

In one aspect, the present disclosure provides a method for detecting a head and neck cancer of the oral cavity or throat in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-4327, hsa-miR-939, hsa-miR-663, hcmv-miR-UL70-3p, hsa-miR-3195, hsa-miR-1268, hsa-miR-3648, hsa-miR-720, hsa-miR-92b, hsa-miR-1237, hsa-miR-1225-5p, hsa-miR-4270, hsa-miR-1202, hsa-miR-1207-5p, hsa-miR-149, hsa-let-7f-1, hsa-miR-23c and hsa-miR-1539, and wherein the level of expression of the at least one miRNA in the biological sample relative to the level of expression of the at least one miRNA in one or more cancer-free reference samples is indicative of the presence of a head and neck cancer of the oral cavity or throat of the subject.

The head and neck cancer of the oral cavity or throat is typically an oral, oropharyngeal, pharyngeal or laryngeal cancer. More typically the cancer is a squamous cell carcinoma.

In particular embodiments the biological sample obtained from the subject is a blood sample, more typically a serum sample. Typically the reference sample(s) is a blood sample, more typically a serum sample. The reference sample(s) may be derived from one or more individuals known not to have a head and neck cancer of the oral cavity or throat.

In one embodiment an increase in expression of one or more miRNAs selected from hsa-let-7a, hsa-miR-16, hsa-miR-451, hsa-miR-486-5p, hsa-miR-92a-3p, hsa-miR-4327, hsa-miR-939, hsa-miR-663, hcmv-miR-UL70-3p, hsa-miR-3195, hsa-miR-1268, hsa-miR-3648 and hsa-miR-720 in the biological sample obtained from the subject relative to the reference sample(s) is indicative of the presence of a head and neck cancer of the oral cavity or throat of the subject. In a particular embodiment the miRNAs are two or more, three or more, four or more or five or more selected from hsa-let-7a, hsa-miR-16, hsa-miR-21, hsa-miR-451, hsa-miR-486-5p and hsa-miR-92a-3p.

In another embodiment a decrease in expression of one or more miRNAs selected from hsa-miR-92b, hsa-miR-1237, hsa-miR-1225-5p, hsa-miR-4270, hsa-miR-1202, hsa-miR-1207-5p, hsa-miR-149, hsa-let-7f-1, hsa-miR-23c and hsa-miR-1539 in the biological sample obtained from the subject relative to the reference sample(s) is indicative of the presence of a head and neck cancer of the oral cavity or throat of the subject.

In another aspect, the present disclosure provides a method for detecting oral cancer in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-let-7b, hsa-miR-15b, hsa-miR-3195, hsa-miR-1268, hsa-miR-3648, hsa-miR-320c, hsa-miR-365, hsa-miR-1238, hsa-miR-191, hsa-miR-1281, hsa-let-7f-1, hsa-miR-149, hsa-miR-23c, hsa-miR-1539, hsa-miR-1225-3p, hsa-miR-3676 and hsa-miR-92b, and wherein the level of expression of the at least one miRNA in the biological sample relative to the level of expression of the at least one miRNA in one or more cancer-free reference samples is indicative of the presence of oral cancer in the subject.

Typically the oral cancer is a squamous cell carcinoma.

In particular embodiments the biological sample obtained from the subject is a blood sample, more typically a serum sample. Typically the reference sample(s) is a blood sample, more typically a serum sample. The reference sample(s) may be derived from one or more individuals known not to have oral cancer.

In one embodiment an increase in expression of one or more miRNAs selected from let-7a, miR-16, miR-21, miR-451, miR-486-5p, miR-92a-3p, hsa-let-7b, hsa-miR-15b, hsa-miR-3195, hsa-miR-1268, hsa-miR-3648, hsa-miR-320c, and hsa-miR-365 in the biological sample obtained from the subject relative to the reference sample(s) is indicative of the presence of oral cancer in the subject. In a particular embodiment the miRNAs are two or more, three or more, four or more or five or more selected from hsa-let-7a, hsa-miR-16, hsa-miR-21, hsa-miR-451, hsa-miR-486-5p and hsa-miR-92a-3 p.

In another embodiment a decrease in expression of one or more miRNAs selected from hsa-miR-1238, hsa-miR-191, hsa-miR-1281, hsa-let-7f-1, hsa-miR-149, hsa-miR-23c, hsa-miR-1539, hsa-miR-1225-3p, hsa-miR-3676 and hsa-miR-92b in the biological sample obtained from the subject relative to the reference sample(s) is indicative of the presence of oral cancer in the subject.

In one embodiment the method comprises determining the expression of two or more of hsa-let-7a, hsa-miR-15b, hsa-miR-486-5p, hsa-miR-451, hsa-miR-16, hsa-miR-365 and hsa-miR-21 in the biological sample, wherein an increase in expression of said miRNAs relative to their expression in one or more cancer-free reference samples is indicative or oral cancer in the subject.

In another aspect, the present disclosure provides a method for detecting oropharyngeal cancer in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-4327, hsa-miR-939, hsa-miR-720, hcmv-miR-UL70-3p, hsa-miR-663, hsa-miR-3195, hsa-miR-1268, hsa-miR-3648, hsa-miR-1237, hsa-miR-92b, hsa-miR-23c, hsa-miR-149, hsa-miR-4310, hsa-let-7f-1, hsa-miR-1539, hsa-miR-1225-3p, hsa-miR-3676 and hsa-miR-766, and wherein the level of expression of the at least one miRNA in the biological sample relative to the level of expression of the at least one miRNA in one or more cancer-free reference samples is indicative of the presence of oropharyngeal cancer in the subject.

Typically the oropharyngeal cancer is a squamous cell carcinoma.

In particular embodiments the biological sample obtained from the subject is a blood sample, more typically a serum sample. Typically the reference sample(s) is a blood sample, more typically a serum sample. The reference sample(s) may be derived from one or more individuals known not to have oropharyngeal cancer.

In one embodiment an increase in expression of one or more miRNAs selected from hsa-let-7a, hsa-miR-16, hsa-miR-21, hsa-miR-451, hsa-miR-486-5p, hsa-miR-92a-3p, hsa-miR-4327, hsa-miR-939, hsa-miR-720, hcmv-miR-UL70-3p, hsa-miR-663, hsa-miR-3195, hsa-miR-1268 and hsa-miR-3648 in the biological sample obtained from the subject relative to the reference sample(s) is indicative of the presence of oropharyngeal cancer in the subject. In a particular embodiment the miRNAs are two or more, three or more, four or more or five or more selected from hsa-let-7a, hsa-miR-16, hsa-miR-21, hsa-miR-451, hsa-miR-486-5p and hsa-miR-92a-3p.

In another embodiment a decrease in expression of one or more miRNAs selected from hsa-miR-1237, hsa-miR-92b, hsa-miR-23c, hsa-miR-149, hsa-miR-4310, hsa-let-7f-1, hsa-miR-1539, hsa-miR-1225-3p, hsa-miR-3676 and hsa-miR-766 in the biological sample obtained from the subject relative to the reference sample(s) is indicative of the presence of oropharyngeal cancer in the subject.

In another aspect, the present disclosure provides a method for detecting pharyngeal or laryngeal cancer in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-2861, hsa-miR-1915, hsa-miR-766, hsa-miR-933, kshv-miR-K12-3, hsa-miR-33b, hsa-miR-720, hsa-miR-1225-5p, hsa-miR-4270, hsa-miR-1202, hsa-miR-1207-5p, hsa-miR-1249, hsv2-miR-H16, hsa-miR-4298, hsa-miR-1237 and hsa-miR-92b, and wherein the level of expression of the at least one miRNA in the biological sample relative to the level of expression of the at least one miRNA in one or more cancer-free reference samples is indicative of the presence of pharyngeal or laryngeal cancer.

Typically the pharyngeal or laryngeal cancer is a squamous cell carcinoma.

In particular embodiments the biological sample obtained from the subject is a blood sample, more typically a serum sample. Typically the reference sample(s) is a blood sample, more typically a serum sample. The reference sample(s) may be derived from one or more individuals known not to have pharyngeal or laryngeal cancer.

In one embodiment an increase in expression of one or more miRNAs selected from hsa-let-7a, hsa-miR-16, hsa-miR-21, hsa-miR-451, hsa-miR-486-5p, hsa-miR-92a-3p, hsa-miR-2861, hsa-miR-1915, hsa-miR-766, hsa-miR-933, kshv-miR-K12-3, hsa-miR-33b and hsa-miR-720 in the biological sample obtained from the subject relative to the reference sample(s) is indicative of the presence of pharyngeal or laryngeal cancer in the subject. In a particular embodiment the miRNAs are two or more, three or more, four or more or five or more selected from hsa-let-7a, hsa-miR-16, hsa-miR-21, hsa-miR-451, hsa-miR-486-5p and hsa-miR-92a-3p.

In another embodiment a decrease in expression of one or more miRNAs selected from hsa-miR-1225-5p, hsa-miR-4270, hsa-miR-1202, hsa-miR-1207-5p, hsa-miR-1249, hsv2-miR-H6, hsa-miR-4298, hsa-miR-1237 and hsa-miR-92b in the biological sample obtained from the subject relative to the reference sample(s) is indicative of the presence of pharyngeal or laryngeal cancer in the subject.

In another aspect, the present disclosure provides a method for detecting a head and neck cancer of the oral cavity or throat in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-4327, hsa-miR-939, hsa-miR-663, hcmv-miR-UL70-3p, hsa-miR-3195, hsa-miR-1268, hsa-miR-3648, hsa-miR-720, hsa-let-7b, hsa-miR-15b, hsa-miR-320c, hsa-miR-365, hsa-miR-2861, hsa-miR-1915, hsa-miR-766, hsa-miR-933, kshv-miR-K12-3 and hsa-miR-33b, and wherein an increase in the level of expression of the at least one miRNA in the biological sample relative to the level of expression of the at least one miRNA in one or more cancer-free reference samples is indicative of the presence of a head and neck cancer of the oral cavity or throat of the subject.

The head and neck cancer of the oral cavity or throat is typically an oral, oropharyngeal, pharyngeal or laryngeal cancer. More typically the cancer is a squamous cell carcinoma.

An increase in expression of hsa-miR-3195, hsa-miR-1268, hsa-miR-486-5p, hsa-miR-3648 and/or hsa-miR-451 may be indicative of oral or oropharyngeal cancer. An increase in the expression of hsa-let-7a, hsa-miR-16, hsa-miR-21, hsa-miR-451, hsa-miR-486-5p, hsa-miR-92a-3p, hsa-let-7b, hsa-miR-15b, hsa-miR-320c, and/or hsa-miR-365 may be indicative of oral cancer. An increase in the expression of hsa-miR-4327, hsa-miR-939, hcmv-miR-UL70-3p and/or hsa-miR-663 may be indicative of oropharyngeal cancer. An increase in the expression of hsa-miR-2861, hsa-miR-1915, hsa-miR-766, hsa-miR-933, kshv-miR-K12-3, hsa-miR-33b and/or hsa-miR-720 may be indicative of pharyngeal or laryngeal cancer.

In another aspect, the present disclosure provides a method for detecting a head and neck cancer of the oral cavity or throat in a subject, the method comprising executing the step of determining the expression of at least one miRNA in a biological sample obtained from a subject, wherein the at least one miRNA is selected from the group consisting of hsa-miR-92b, hsa-miR-1237, hsa-miR-1225-5p, hsa-miR-4270, hsa-miR-1202, hsa-miR-1207-5p, hsa-miR-149, hsa-let-7f-1, hsa-miR-23c, hsa-miR-1238, hsa-miR-191, hsa-miR-1281, hsa-miR-4310, hsa-miR-766, hsa-miR-1249, hsv2-miR-H6, hsa-miR-4298 and hsa-miR-1539, and wherein a decrease in the level of expression of the at least one miRNA in the biological sample relative to the level of expression of the at least one miRNA in one or more cancer-free reference samples is indicative of the presence of a head and neck cancer of the oral cavity or throat of the subject.

The head and neck cancer of the oral cavity or throat is typically an oral, oropharyngeal, pharyngeal or laryngeal cancer. More typically the cancer is a squamous cell carcinoma.

A decrease in the expression of hsa-miR-92b may be indicative of oral, oropharyngeal, pharyngeal or laryngeal cancer. A decrease in the expression of hsa-miR-149, hsa-let-7f-1, hsa-miR-23c, hsa-miR-3676 and/or hsa-miR-1539 may be indicative of oral or oropharyngeal cancer. A decrease in the expression of hsa-miR-1237 may be indicative of oropharyngeal, pharyngeal or laryngeal cancer. A decrease in the expression of hsa-miR-1238, hsa-miR-191 and/or hsa-miR-1281 may be indicative of oral cancer. A decrease in the expression of hsa-miR-4310 and/or hsa-miR-766 may be indicative of oropharyngeal cancer. A decrease in the expression of hsa-miR-4270, hsa-miR-1202, hsa-miR-1207-5p, hsa-miR-1249, hsv2-miR-H6, hsa-miR-4298 and/or hsa-miR-1225-5p may be indicative of pharyngeal or laryngeal cancer.

In another aspect, the present disclosure provides a method for detecting oral cancer in a subject, the method comprising executing the step of determining the expression of two or more miRNAs in a biological sample obtained from a subject, wherein the two or more miRNAs are selected from the group consisting of hsa-let-7a, hsa-miR-16, hsa-miR-21, hsa-miR-451, hsa-miR-486-5p, hsa-miR-92a-3p, hsa-miR-15b and hsa-miR-365, and wherein an increase in the level of expression of the two or more miRNAs in the biological sample relative to the level of expression of the two or more miRNAs in one or more cancer-free reference samples is indicative of the presence of oral cancer in the subject.

In particular embodiments, the method comprises determining the expression of three or more, four or more, five or more of six or more of said miRNAs. In one exemplary embodiment, the method comprises determining the expression of hsa-let-7a, hsa-miR-15b, hsa-miR-486-5p, hsa-miR-451, hsa-miR-16 and hsa-miR-365. In a further exemplary embodiment, the method comprises determining the expression of hsa-let-7a, hsa-miR-16, hsa-miR-1, hsa-miR-451, hsa-miR-486-5p and hsa-miR-92a-3p. In a further exemplary embodiment, the method comprises determining the expression of hsa-miR-16, hsa-miR-486-5p and hsa-miR-92a-3p.

In another aspect, the present disclosure provides a method for detecting an oral cancer in a subject, the method comprising executing the step of determining the expression of the miRNAs hsa-miR-16, hsa-miR-486-5p and hsa-miR-92a-3p in a biological sample obtained from a subject, wherein an increase in the level of expression of said miRNAs in the biological sample relative to the level of expression of said miRNAs in one or more cancer-free reference samples is indicative of the presence of oral cancer in the subject.

In an embodiment, the oral cancer is oral squamous cell carcinoma.

In another aspect, the present disclosure provides a method for predicting the probability of survival of a subject having oral cancer, the method comprising executing the step of determining the expression of the miRNAs hsa-let-7a, hsa-miR-16, hsa-miR-21, hsa-miR-451, hsa-miR-486-5p and hsa-miR-92a-3p in a biological sample obtained from a subject having oral cancer, wherein an increase in the level of expression of said miRNAs in the biological sample relative to the level of expression of said miRNAs in one or more cancer-free reference samples is indicative of a reduced likelihood of survival of the individual beyond about four years.

In an embodiment, the oral cancer is oral squamous cell carcinoma.

In accordance with the above described aspects and embodiments, expression data or profiles for the selected miRNAs may be subjected to one or more statistical analyses to determine a miRNA signature profile, thereby facilitating the diagnostic or prognostic method. The statistical analysis may comprise, for example, logistical regression; logistical regression with k-fold validation, machine learning or machine learning with k-fold validation. The statistical analysis may comprise determining one or more of ΔCt or Cq values for the selected miRNAs.

By way of example only, where the miRNAs comprise or consist of hsa-miR-16, hsa-miR-486-5p and hsa-miR-92a-3p, the probability of being diagnosed with an oral cancer, optionally oral squamous cell carcinoma, may be determined according to the formula:

Logit [p=OC] wherein Log p/1−p=(−)59.5+0.73×Cq[hsa-miR-16]+(−)2.23×Cq[hsa-miR-92a-3p]+3.27×Cq[hsa-miR-486-5p]

By way of example only, where the miRNAs comprise or consist of hsa-let-7a, hsa-miR-16, hsa-miR-21, hsa-miR-451, hsa-miR-486-5p and hsa-miR-92a-3p, the likelihood of reduced survival of the subject may be determined according to the formula:

let-7a×(−0.4729)+Cq[hsa-miR-451]×0.5305+Cq[hsa-miR-16]×0.2646+Cq[hsa-miR-21]×(−0.2593)+Cq[hsa-miR-92a-3p]×(−0.6423)+Cq[hsa-miR-486-5p]×0.4272

Also provided herein are kits for use in screening for head and neck cancers of the oral cavity and throat, wherein the kits comprise one or more reagents for determining the expression of one or more miRNAs as defined in the above aspects and embodiments.

Also provided herein is a computer system or apparatus, configured to aid in the detection or diagnosis of a head and neck cancer of the oral cavity or throat, wherein computer software is employed to analyse data relating to the expression of one or more miRNAs as defined in the above aspects and embodiments, in a biological sample obtained from a subject and to provide a diagnostic prediction with respect to the subject. Typically, the compute software is also employed to compare said data to data relating to the expression of the one or more miRNAs in one or more cancer-free reference samples.

Also provided herein is a method for selecting a subject for treatment for a head and neck cancer of the oral cavity or throat, the method comprising:

(a) executing a step of determining the level of expression of one or more miRNAs as defined in the above aspects and embodiments in a biological sample obtained from a subject, wherein the level of expression of the at least one miRNA in the biological sample relative to the level of expression of the at least one miRNA in one or more cancer-free reference samples is indicative of the presence of a head and neck cancer of the oral cavity or throat of the subject; and

(b) selecting a subject, identified in (a) as having a head and neck cancer of the oral cavity or throat, for treatment for said cancer.

Also provided herein is a protocol for monitoring the efficacy of a therapeutic treatment for a head and neck cancer of the oral cavity or throat, the protocol comprising:

(a) obtaining from a subject a first biological sample, wherein the first biological sample is obtained before or after commencement of treatment;

(b) obtaining from the same subject a second biological sample, wherein the second biological sample is obtained at a time point after commencement of treatment and after the first biological sample is obtained;

(c) executing the step of measuring for expression of at least one miRNA in the first and second biological samples, wherein the at least one miRNA is as defined in the above aspects and embodiments; and

(d) comparing the expression of the at least one miRNA in the first biological sample with the expression of the same at least one miRNA in the second biological sample; wherein a change in the expression of the at least one miRNA between the first and second biological samples is indicative of whether or not the therapeutic treatment is effective.

The protocol may further comprise obtaining and executing steps in respect of a third or subsequent sample.

The above described protocol may be used in the screening of candidate agents for treating the cancer.

### BRIEF DESCRIPTION OF THE DRAWINGS

Embodiments of the disclosure are described herein, by way of non-limiting example only, with reference to the following drawings.

FIG. 1. Maximally over expressed and under expressed miRNAs with a p-value of less than 0.000001 in pooled head and neck squamous cell carcinoma samples, as identified by volcano plot analysis.

FIG. 2. Maximally over expressed and under expressed miRNAs with a p-value of less than 0.000001 in pooled oral squamous cell carcinoma samples, as identified by volcano plot analysis.

FIG. 3. Gene ontology mapping of dysregulated miRNAs in pooled oral squamous cell carcinoma samples. Gene ontology categories were divided into (A) biological processes and (B) molecular function.

FIG. 4. Expression levels (as determined by qPCR) of miRNAs hsa-let-7a, hsa-miR-15b, hsa-miR-16, hsa-miR-21, hsa-miR-365, hsa-miR-451 and hsa-miR-486-5p in pooled samples derived from individuals having oral squamous cell carcinoma (cancer) and in pooled samples from cancer-free individuals (healthy).

FIG. 5. Maximally over expressed and under expressed miRNAs with a p-value of less than 0.000001 in pooled orophayngeal squamous cell carcinoma samples, as identified by volcano plot analysis.

FIG. 6. Gene ontology mapping of dysregulated miRNAs in pooled orophayngeal squamous cell carcinoma samples. Gene ontology categories were divided into (A) biological processes and (B) molecular function.

FIG. 7. Maximally over expressed and under expressed miRNAs with a p-value of less than 0.000001 in pooled pharyngeal/laryngeal squamous cell carcinoma samples, as identified by volcano plot analysis.

FIG. 8. Gene ontology mapping of dysregulated miRNAs in pooled pharyngeal/laryngeal squamous cell carcinoma samples. Gene ontology categories were divided into (A) biological processes and (B) molecular function.

FIG. 9. Expression levels (as determined by qPCR) of miRNAs hsa-miR-365, hsa-let-7a, hsa-miR-486-5p, hsa-miR-451, hsa-miR-15b and hsa-miR-16 in both hemolysed and non-hemolysed pooled samples derived from individuals having oral squamous cell carcinoma (cancer) and in pooled samples from cancer-free individuals (healthy).

FIG. 10. Expression levels (as determined by RNAmp assay) of miRNAs let-7a, miR-16, miR-21, miR-451, miR-486-5p and miR-92a-3p in serum from patients with head and neck cancer and healthy patients. A. Expression levels of each miRNA individually. B Average expression levels of combined miRNA. Yellow boxes represent cancer samples, and grey boxes represent healthy samples.

FIG. 11. Box Plot categorisation of 6 individual biomarker Cq Values across oral squamous cell carcinoma samples and healthy controls. The bold middle line of each plot signifies the median of the data set. The lower line represents the 25th percentile, i.e 25% of the Ct 92 cohort had a value of about Ct 25 or less. The top line is the cut-off for the 75th percentile. Taken together, these two whiskers represent 100% of real data while points not within were considered outliers.

FIG. 12. The miRNA diagnostic classifier Tri_{miR} was established with Logistical Regression modelling with an AUC 0.9 [0.734−0.978], a sensitivity of 91.3 and specificity of 85.7. The predicted probability of being diagnosed as any stage of oral squamous cell carcinoma by Tri_{miR} was calculated by: Logit [p=OC] whereby Log p/1−p=(−)59.5+0.73×hsa-miR-16+(−)2.23×hsa-miR-92a-3p+3.27×hsa-miR-486-5p. In this equation, the miRNA symbol was substituted with the Cq value.

FIG. 13. The polygenic 6_{miR} signature was associated with a low survival probability upon diagnosis. A personalised linear score ranked an individual having oral squamous cell carcinoma with a risk of survival. Using eight predictor variables (age, sex and abundance of the six miRNAs) the index was: let−7a×(−0.4729)+hsa-miR-451×0.5305+hsa-miR-16×0.2646+hsa-miR-21×(−0.2593)+-hsa-miR-92a-3p×(−0.6423)+hsa-miR-486-5p×0.4272. A risk score of over 4.8 indicated a higher chance of death upon initial diagnosis.

FIG. 14. Expression levels (as determined by RNAmp assay) of miRNAs let-7a, miR-16, miR-21, miR-451, miR-486-5p and miR-92a-3p in serum from patients with head and neck cancer, where the serum contains varying levels of hemolysis as represented by varying amounts of free haemoglobin.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a miRNA” includes a single miRNA, as well as two or more miRNAs.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, “microRNA” or “miRNA” refers to a non-coding RNA, typically between about 18 and 25 nucleotides in length that hybridizes to and regulates the expression of a coding RNA. In certain embodiments, a miRNA is the product of cleavage of a precursor (pre-miRNA), for example by the enzyme Dicer. As used herein, “pre-miRNA” refers to a non-coding RNA having a hairpin structure, which contains a miRNA. Typically the term “pre-miRNA” refers to a precursor molecule, the processing and cleavage of which gives rise to a mature miRNA. In certain embodiments, a pre-miRNA is the product of cleavage of a pri-miR by a double-stranded RNA-specific ribonuclease.

The term “subject” as used herein refers to mammals and includes humans, primates, livestock animals (e.g. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human.

In recent years there has been much interest in non-coding RNAs (ncRNAs), the best understood of which are microRNAs (miRNAs). miRNAs are a class of short, endogenous, single-stranded, non-coding RNA molecules that bind with imperfect complementarity to the 3′ untranslated regions (3′-UTRs) of target mRNAs. miRNAs are initially transcribed as long primary transcripts (pri-miRNAs or pri-miRs). These are typically processed in the nucleus by the Drosha-DGCR8 complex, producing a 60-70 nucleotide (nt) stem loop structure known as precursor miRNA (pre-miRNA). The pre-miRNA is then exported to the cytoplasm and further processed into an intermediate miRNA duplex before association with the RNA-induced silencing complex (RISC) and maturation to single stranded miRNA. Mature miRNAs interact with sites of imperfect complementarity in 3′ untranslated regions (UTRs) of target mRNAs. These targeted transcripts subsequently undergo accelerated turnover and translational down regulation.

Although miRNAs represent less than 0.1% of the entire mammalian transcriptome, they can control up to two thirds of gene expression in mammalian cells. There is now overwhelming evidence that many miRNAs are dysregulated in common cancers such as those originating in the breast, lung, colon, liver, and the prostate. They are regarded as key regulators in the process of tumourigenesis and many studies have suggested the use of specific miRNAs as potential biomarkers for cancer.

Circulating miRNAs have been detected in most human bodily fluids, including plasma, serum, saliva, sweat, tears, breast milk and urine. As such miRNA levels can be readily determined using non-invasive techniques using standard techniques and methods well known to those skilled in the art. Circulating miRNAs are also extremely stable and are RNASE-resistant. These characteristics make circulating miRNAs excellent candidates as biomarkers of disease.

The present disclosure is predicated on the inventors' surprising findings that specific miRNAs and groups of miRNAs are specifically over expressed (up regulated) or under expressed (down regulated) in oral, oropharyngeal, pharyngeal and laryngeal cancers. These cancers are referred to herein collectively as head and neck cancers of the oral cavity or throat. The miRNAs can be rapidly detected in whole blood or blood serum.

The present disclosure therefore provides, for the first time, a suite of biomarkers suitable for the rapid and early detection and diagnosis of a range of head and neck cancers, thereby enabling appropriate treatment and patient management strategies to be put into place before progression of the disease to later stages less amenable to treatment.

The present disclosure thereby also provides means of improving the prognosis of sufferers of head and neck cancers of the oral cavity and throat by early detection and diagnosis using the biomarkers and suites of biomarkers disclosed herein, and thus early intervention.

In broad terms, disclosed herein is a method for detecting a head and neck cancer of the oral cavity or throat in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-4327, hsa-miR-939, hsa-miR-663, hcmv-miR-UL70-3p, hsa-miR-3195, hsa-miR-1268, hsa-miR-3648, hsa-miR-720, hsa-miR-92b, hsa-miR-1237, hsa-miR-1225-5p, hsa-miR-4270, hsa-miR-1202, hsa-miR-1207-5p, hsa-miR-149, hsa-let-7f-1, hsa-miR-23c and hsa-miR-1539, and wherein the level of expression of the at least one miRNA in the biological sample relative to the level of expression of the at least one miRNA in one or more cancer-free reference samples is indicative of the presence of a head and neck cancer of the oral cavity or throat of the subject.

Also in broad terms, disclosed herein is a method for detecting oral cancer in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-let-7b, hsa-miR-15b, hsa-miR-3195, hsa-miR-1268, hsa-miR-3648, hsa-miR-320c, hsa-miR-365, hsa-miR-1238, hsa-miR-191, hsa-miR-1281, hsa-let-7f-1, hsa-miR-149, hsa-miR-23c, hsa-miR-1539, hsa-miR-1225-3p, hsa-miR-3676 and hsa-miR-92b, and wherein the level of expression of the at least one miRNA in the biological sample relative to the level of expression of the at least one miRNA in one or more cancer-free reference samples is indicative of the presence of oral cancer in the subject.

Also in broad terms, disclosed herein is a method for detecting oropharyngeal cancer in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-4327, hsa-miR-939, hsa-miR-720, hcmv-miR-UL70-3p, hsa-miR-663, hsa-miR-3195, hsa-miR-1268, hsa-miR-3648, hsa-miR-451, hsa-miR-1237, hsa-miR-92b, hsa-miR-23c, hsa-miR-149, hsa-miR-4310, hsa-let-7f-1, hsa-miR-1539, hsa-miR-1225-3p, hsa-miR-3676 and hsa-miR-766, and wherein the level of expression of the at least one miRNA in the biological sample relative to the level of expression of the at least one miRNA in one or more cancer-free reference samples is indicative of the presence of oropharyngeal cancer in the subject.

Also in broad terms, disclosed herein is a method for detecting pharyngeal or laryngeal cancer in a subject, the method comprising executing the step of determining the expression of at least one miRNA in a biological sample obtained from a subject, wherein the at least one miRNA is selected from the group consisting of hsa-let-7a, hsa-miR-16, hsa-miR-21, hsa-miR-451, hsa-miR-486-5p, hsa-miR-92a-3p, hsa-miR-2861, hsa-miR-1915, hsa-miR-766, hsa-miR-933, kshv-miR-K12-3, hsa-miR-33b, hsa-miR-720, hsa-miR-1225-5p, hsa-miR-4270, hsa-miR-1202, hsa-miR-1207-5p, hsa-miR-1249, hsv2-miR-H6, hsa-miR-4298, hsa-miR-1237 and hsa-miR-92b, and wherein the level of expression of the at least one miRNA in the biological sample relative to the level of expression of the at least one miRNA in one or more cancer-free reference samples is indicative of the presence of pharyngeal or laryngeal cancer.

In some embodiments disclosed herein, the method comprises measuring the expression of at least two miRNAs (e.g., 2, 3, 4, 5, 6, 7 or more) selected from the group of miRNAs disclosed herein. The use of combinations of miRNA biomarkers can serve to improve the sensitivity and/or specificity of cancer detection and diagnosis. Combinations of any of the miRNAs disclosed herein may be employed. For example, for the detection of oral cancer, the method may comprise executing the step of determining the expression of two or more miRNAs in a biological sample obtained from a subject, wherein the two or more miRNAs are selected from the group consisting of hsa-let-7a, hsa-miR-15b, hsa-miR-486-5p, hsa-miR-451, hsa-miR-16, hsa-miR-365 and hsa-miR-21, and wherein an increase in the level of expression of the two or more miRNAs in the biological sample relative to the level of expression of the two or more miRNAs in one or more cancer-free reference samples is indicative of the presence of oral cancer in the subject. In another example, for the detection of head and neck cancer of the oral cavity or throat in a subject, the method may comprise executing the step of determining the expression of two or more miRNAs in a biological sample obtained from a subject, wherein the two or more miRNAs are selected from the group consisting of hsa-let-7a, hsa-miR-16, hsa-miR-21, hsa-miR-486-5p, hsa-miR-451 and hsa-miR-92a-3p, wherein an increase in the level of expression of the two or more miRNAs in a biological sample relative to the level of expression of the two or more miRNAs in one or more cancer-free reference samples is indicative of the presence of head and neck cancer of the oral cavity or throat in the subject.

In a particular embodiment, the present disclosure provides a method for detecting an oral cancer, optionally oral squamous cell carcinoma, in a subject, the method comprising executing the step of determining the expression of the miRNAs hsa-miR-16, hsa-miR-486-5p and hsa-miR-92a-3p in a biological sample obtained from a subject, wherein an increase in the level of expression of said miRNAs in the biological sample relative to the level of expression of said miRNAs in one or more cancer-free reference samples is indicative of the presence of oral cancer in the subject.

In another particular embodiment, the present disclosure provides a method for predicting the probability of survival of a subject having oral cancer, the method comprising executing the step of determining the expression of the miRNAs hsa-let-7a, hsa-miR-16, hsa-miR-21, hsa-miR-451, hsa-miR-486-5p and hsa-miR-92a-3p in a biological sample obtained from a subject having oral cancer, wherein an increase in the level of expression of said miRNAs in the biological sample relative to the level of expression of said miRNAs in one or more cancer-free reference samples is indicative of a reduced likelihood of survival of the individual beyond about four years.

The term “expression” is used herein in its broadest context to denote a measurable presence of the biomarker miRNA. As described hereinbelow, a variety of methods of determining or measuring expression of miRNAs are contemplated. In some embodiments, measuring the expression of a miRNA comprises determining the level of the miRNA. As used herein the terms “level” and “amount” may be used interchangeably to refer to a quantitative amount, a semi-quantitative amount, a relative amount, a concentration, or the like. Thus, these terms encompass absolute or relative amounts or concentrations of a miRNA in a sample, including levels in a population of subjects represented as mean levels and standard deviations.

The sequences of the mature miRNAs the subject of the present disclosure, and of corresponding pre-miRNAs, are publicly available through the miRBase database (www.mirbase.org). Sequences of the mature miRNAs disclosed herein are also provided in the Sequence Listing appearing at the end of this specification, according to the following Tables 1 and 2.

TABLE 1

| miRNA | SEQ ID NO. |
|---|---|
| hsa-let-7a-5p | 1 |
| hsa-let-7b-5p | 2 |
| hsa-miR-15b-5p | 3 |
| hsa-miR-16-5p | 4 |
| hsa-miR-21-5p | 5 |
| hsa-miR-33b-5p | 6 |
| hcmv-miR-UL70-3p | 7 |
| hsa-miR-320c | 8 |
| hsa-miR-365-5p | 9 |
| hsa-miR-451a | 10 |
| hsa-miR-486-5p | 11 |
| hsa-miR-663a | 12 |
| hsa-miR-720 | 13 |
| hsa-miR-766-5p | 14 |
| hsa-miR-933 | 15 |
| hsa-miR-939-5p | 16 |
| hsa-miR-1268a | 17 |
| hsa-miR-1915-5p | 18 |
| hsa-miR-2861 | 19 |
| hsa-miR-3195 | 20 |
| hsa-miR-3648 | 21 |
| hsa-miR-4327 | 22 |
| kshv-miR-K12-3-5p | 23 |

TABLE 2

| miRNA | SEQ ID NO. |
|---|---|
| hsa-let-7f-1-5p | 24 |
| hsa-miR-23c | 25 |
| hsa-miR-92b-5p | 26 |
| hsa-miR-149-5p | 27 |
| hsa-miR-191-5p | 28 |
| hsa-miR-766-5p | 29 |
| hsa-miR-1202 | 30 |
| hsa-miR-1207-5p | 31 |
| hsa-miR-1225-3p | 32 |
| hsa-miR-1225-5p | 33 |
| hsa-miR-1237-5p | 34 |
| hsa-miR-1238-5p | 35 |
| hsa-miR-1249-5p | 36 |
| hsa-miR-1281 | 37 |
| hsa-miR-1539 | 38 |
| hsa-miR-3676 | 39 |
| hsa-miR-4270 | 40 |
| hsa-miR-4298 | 41 |
| hsa-miR-4310 | 42 |
| hsv2-miR-H6-5p | 43 |
| hsa-miR-92a-3p | 44 |

miRNAs to be assessed in accordance with the present disclosure may be obtained from any suitable biological sample, and it is well within the skill of those in the art to determine what type of sample is most appropriate for determining the expression levels of any particular miRNA(s) and for detecting a particular cancer. The biological sample may be any sample in which the expression of the biomarker miRNA(s) can be detected or measured for the purpose of identifying the presence (or absence) of a head and neck cancer of the oral cavity or throat in a subject. Suitable biological samples can be determined by persons skilled in the art, illustrative examples of which include blood, serum, plasma, saliva, urine, tears, peritoneal fluid, ascitic fluid, breast fluid, breast milk, lymph fluid, cerebrospinal fluid or mucosa secretion. In particular embodiments disclosed herein the biological sample comprises whole blood or serum.

The biological sample may be processed and analyzed for the purpose of determining the presence of a head and neck cancer of the oral cavity or throat in accordance with the present disclosure, almost immediately following collection (i.e., as a fresh sample), or it may be stored for subsequent analysis. If storage of the biological sample is desired or required, it would be understood by persons skilled in the art that it should ideally be stored under conditions that preserve the integrity of the biomarker of interest within the sample (e.g., at −80° C.).

Typically, miRNA detection and determination of expression requires isolation of nucleic acid from a sample. Nucleic acids, including RNA and specifically miRNA, can be isolated using any suitable technique known in the art. For example, phenol-based isolation procedures can recover RNA species in the 10-200-nucleotide range (e.g., precursor and mature miRNAs). Extraction procedures such as those using Trizol™ or Tri-Reagent™ can be used to purify all RNAs, large and small, and are efficient methods for isolating total RNA from biological samples that contain miRNAs. Any number of suitable RNA extraction techniques and commercially available RNA extraction kits (e.g. Qiagen RNeasy® kits) are well known to those skilled in the art and may be employed in accordance with the present disclosure.

Any method of detecting and measuring miRNA expression in sample can be used in the methods disclosed herein, with illustrative examples described below. In particular embodiments, determining or measuring the expression of a miRNA comprises determining or measuring the level of the mature miRNA. Alternatively, the expression of the corresponding pre-miRNA or encoding gene may be determined or measured.

In some embodiments biochip-based techniques, such as microarrays, may be desirable for determining and measuring expression (such as are described in Hacia et al., 1996, Nature Genetics 14: 441-447). By tagging nucleic acids with oligonucleotides or using fixed probe arrays, one can employ biochip technology to segregate target molecules as high-density arrays and screen these molecules on the basis of hybridization. The design of suitable nucleic acid probes or oligonucleotides is well within the capabilities and expertise of those skilled in the art. Microarrays can be fabricated using a variety of technologies and microarray analysis of miRNA can be accomplished according to any method known in the art. Several types of microarrays, known to those skilled in the art, can be employed including spotted oligonucleotide microarrays, pre-fabricated oligonucleotide microarrays, long oligonucleotide arrays and short oligonucleotide arrays.

Particles (e.g. beads) in suspension or in planar arrays can also be used as the basis of assays. Biomolecules such as oligonucleotides can be conjugated to the surface of beads to capture miRNAs of interest. A range of detection methods, such as flow cytometric or other suitable imaging technologies, known to persons skilled in the art can then be used for characterization of the beads and detection of miRNA presence.

In particular embodiments of the present disclosure PCR methodologies or other template-dependent amplification techniques may be desirable. For example, any method of PCR that can determine the expression of a nucleic acid molecule, including a miRNA, falls within the scope of the present disclosure. Exemplary PCR methods include, but are not limited to, reverse transcriptase PCR, real time PCR, quantitative PCR (qPCR), quantitative real time PCR (qRT-PCR), and multiplex PCR. The skilled addressee will be able determine the appropriate means of measuring expression in any given circumstance, for any given miRNA(s), without undue burden or experimentation.

Using the known sequences for the miRNAs disclosed herein, specific probes and primers can be designed for use in the detection methods described as appropriate.

It will be understood by those skilled in the art that the method of determining or measuring expression of a miRNA in a biological sample can be quantitative, semi-quantitative or qualitative in nature. For example, quantitative analyses will typically provide a concentration of a miRNA in the sample within an appropriate error margin (eg., mean +/−standard deviation). By contrast, semi-quantitative or qualitative analyses will typically provide an indication of the relative amount of a miRNA in a sample. This may involve a comparison of an amount of miRNA in a first sample with an amount of the same miRNA in a second sample, and making a determination as to the relative amount between the first and second samples.

Methods of the present disclosure may be employed to detect or diagnose a head and neck cancer of the oral cavity or throat in a subject where no diagnosis, or confirmed diagnosis, previously existed. Typically, the expression levels of the miRNAs disclosed herein are compared to reference levels, where the reference levels represent the absence of head and neck cancer of the oral cavity or throat. The reference levels may be from one or more reference samples. In this context the term “reference” or “reference sample” means one or more biological samples from individuals or groups of individuals diagnosed as not having a head and neck cancer of the oral cavity or throat. A “reference sample” may comprise the compilation of data from one or more individuals whose diagnosis as a “reference” or “control” for the purposes of the present disclosure has been confirmed. That is, samples to be used as reference samples or controls need not be specifically or immediately obtained for the purpose of comparison with the sample(s) obtained from a subject under assessment.

Thus, reference levels of miRNAs can be pre-determined using biological samples from a cohort of healthy subjects (i.e. free of head and neck cancer of the oral cavity or throat) to obtain an accurate median or mean. Reference levels can be determined for various samples, such as various cell and tissue types and various body fluids. For the most accurate detection, the reference sample used for comparison comprises the same type of sample as taken from the subject under assessment in the provided methods. Reference levels also can be matched by age, sex or other factor.

Diagnoses made in accordance with embodiments disclosed herein may be correlated with other means of diagnosing head and neck cancers of the oral cavity and throat. Thus, methods of the present disclosure may be used alone or in conjunction with, or as an adjunct to, one or more other diagnostic methods and tests to diagnose head and neck cancers of the oral cavity and throat. Such other diagnostic methods and tests will be well known to those skilled in the art and include, for example, fine needle aspiration biopsy.

### Kits

All essential materials and reagents required for measuring for the expression of the at least one biomarker may be assembled together in a kit. Thus, the present disclosure also provides diagnostic and test kits for detecting or determining the level of expression of one or more of the miRNAs disclosed herein in a biological sample, in order to facilitate the detection or diagnosis of a head and neck cancer of the oral cavity or throat. Such kits typically comprise one or more reagents and/or devices for use in performing the methods disclosed herein. For example, the kits may contain reagents for measuring the expression of one or more miRNA in a biological sample. As such, kits may comprise one or more agents for detecting and to facilitate measurement of miRNAs, including primers, probes or other agents, and/or may comprise suitable reagents for determining or measuring expression of the miRNAs such as diluents, reaction buffers, wash buffers, labelling reagents, enzymes etc). Kits may also comprise the necessary reagents for RNA extraction from samples to be analysed. Some kits may also, for example, include components for making an array comprising oligonucleotides complementary to miRNAs, and thus, may include, for example, a solid support.

Kits for carrying out the methods of the present disclosure may include, in suitable container means comprising, or adapted to receive, reagents required. The container means may include at least one vial, test tube, flask, bottle, syringe and/or other container. The kits may also include means for containing the reagents in close confinement for commercial sale. Such containers may include injection and/or blow-moulded plastic containers.

Kits may also include suitable means to receive a biological sample, one or more containers or vessels for carrying out methods described herein, positive and negative controls, including a reference sample, and instructions for the use of kit components contained therein, in accordance with the methods disclosed herein.

### Therapeutic Regimens

A subject who is identified, in accordance with the methods of the present disclosure described hereinbefore as having a head and neck cancer of the oral cavity or throat, can be selected for treatment, or stratified into a treatment group, wherein an appropriate therapeutic regimen can be adopted or prescribed with a view to treating the cancer.

Thus, in an embodiment, the methods disclosed herein comprise the step of exposing (i.e., subjecting) a subject identified as having a head and neck cancer of the oral cavity or throat to a therapeutic regimen for treating said cancer.

Thus, an aspect of the present disclosure provides a method for selecting a subject for treatment for a head and neck cancer of the oral cavity or throat, the method comprising:

(a) executing a step of determining the level of expression of one or more miRNAs as defined in the above aspects and embodiments in a biological sample obtained from a subject, wherein the level of expression of the at least one miRNA in the biological sample relative to the level of expression of the at least one miRNA in one or more cancer-free reference samples is indicative of the presence of a head and neck cancer of the oral cavity or throat of the subject; and

(b) selecting a subject, identified in (a) as having a head and neck cancer of the oral cavity or throat, for treatment for said cancer.

The nature of therapeutic treatment or regimen to be employed can be determined by persons skilled in the art and will typically depend on factors such as, but not limited to, the age, weight and general health of the subject. Suitable therapeutic treatments and regimens would be known to persons skilled in the art, non-limiting examples of which include chemotherapeutic agents and/or radiotherapy.

As used herein the terms “treating” and “treatment” refer to any and all uses which remedy a condition or symptoms, or disease, or otherwise prevent, hinder, retard, or reverse the progression of a condition or disease or other undesirable symptoms in any way whatsoever. Thus the term “treating” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms.

Without being bound by theory or a particular mode of practice, it also follows from the present disclosure that the methods disclosed herein can be used to monitor the efficacy of treatment of a head and neck cancer of the oral cavity or throat, whereby the expression of one or more miRNAs disclosed herein is determined (e.g., measured) in biological samples obtained from a subject at two or more separate time points, including before commencement of treatment, during the course of treatment and after cessation of treatment, to determine whether said treatment is effective, for example, in inhibiting the progression of the cancer.

Thus, also provided herein is a protocol for monitoring the efficacy of a therapeutic treatment for a head and neck cancer of the oral cavity or throat, the protocol comprising:

(a) obtaining from a subject a first biological sample, wherein the first biological sample is obtained before or after commencement of treatment;

(b) obtaining from the same subject at least a second biological sample, wherein the second biological sample is obtained at a time point after commencement of treatment and after the first biological sample is obtained;

(c) executing the step of measuring for expression of at least one miRNA in the first and second biological samples, wherein the at least one miRNA is as defined in the above aspects and embodiments; and

(d) comparing the expression of the at least one miRNA in the first biological sample with the expression of the same at least one miRNA in the second biological sample;

wherein a change in the expression of the at least one miRNA between the first and second biological samples is indicative of whether or not the therapeutic treatment is effective.

The protocol may further comprise obtaining and executing steps in respect of a third or subsequent sample.

In an embodiment, a change of expression of a miRNA between the first and second (or subsequent) sample may be indicative of an effective therapeutic regimen. Where the protocol disclosed herein indicates that the therapeutic regimen is ineffective (i.e. no change in expression of one or more miRNAs between the first and second, or subsequent, sample), the protocol may further comprises altering or otherwise modifying the therapeutic regimen with a view to providing a more efficacious or aggressive treatment. This may comprise administering to the subject additional doses of the same agent with which they are being treated or changing the dose and/or type of medication.

Also provided herein are screening methods for candidate compounds and compositions as therapeutic agents to treat a head and neck cancer of the oral cavity or throat. For example, a suitable therapeutic agent for a cancer may be obtained by selecting a compound or composition capable of increasing or decreasing the expression level of one or more miRNAs as disclosed herein.

Such methods of screening for a therapeutic agent can be carried out either in vivo or in vitro. For example, a screening method may be performed by administering a candidate compound or composition to a subject, such as a laboratory test animal subject; measuring the expression level of a miRNA in a biological sample from the subject; and selecting a compound or composition that increases or decreases the expression level of the miRNA, as compared to that in a control with which the candidate compound or composition has not been contacted.

Methods for selecting a compound or composition for treating a head and neck cancer of the oral cavity or throat, for monitoring the efficacy of such a treatment or for screening candidate agents may also be employed by, for example: obtaining a biological sample from a subject, such as a laboratory test animal subject; separately maintaining aliquots of the sample in the presence of a plurality of compounds or compositions; comparing expression of a miRNA in each of the aliquots; and selecting one of the compounds or compositions which significantly alters the level of expression of the miRNA in the aliquot containing that compound or composition, relative to the levels of expression of the mRNA in the presence of other compounds or compositions.

It will be appreciated that the above described terms and associated definitions are used for the purpose of explanation only and are not intended to be limiting.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### General Methods

Head and neck squamous cell carcinoma (HNSCC) serum samples were obtained from subjects through informed written consent with approval by the ethics boards at the University of Technology and Royal Prince Alfred Hospital. Serum samples were selected from 52 HNSCC patients with a pathological diagnosis of either oral cavity tumour (n=42; average age 62.1 years; 68% males), oro-pharyngeal tumour (n=5; average age 71.4 years; 60% males), or pharyngeal/laryngeal tumour (n=5; average age 60.8 years; 80% males). Details are provided below in Table 3.

TABLE 3

| Age | Gender | Tumour Site | Tumour Stage |
|---|---|---|---|
| 73 | F | Right Maxilla | 4a |
| 73 | M | Anterior 2/3 of Tongue | 2 |
| 56 | M | Soft Palate | 2 |
| 60 | M | Anterior 2/3 of Tongue | 1 |
| 69 | M | Mandible | 4a |
| 76 | M | Tongue SCC | 2 |
| 66 | F | Partial Lossectomy | 1 |
| 56 | M | Left Parotidectomy | 4a |
| 71 | M | Ventral Surface of Tongue | 3 |
| 32 | M | Hard Palate | 2 |
| 68 | M | Anterior 2/3 of Tongue | 2 |
| 69 | F | Anterior 2/3 of Tongue | 2 |
| 65 | M | Anterior 2/3 of Tongue | 1 |
| 75 | M | Anterior 2/3 of Tongue | 1 |
| 47 | M | Lower Gum | 4 |
| 71 | F | Right Alveolus SCC | 2 |
| 86 | F | Mandibular SCC | 4 |
| 53 | M | Right Tongue SCC | 1 |
| 71 | M | Oral Carcinoma | 2 |
| 79 | M | Retromolar Trigone | 3 |
| 71 | F | Oral Carcinoma (Recurrent | 2 |
| | | SCC) | |
| 76 | M | Tongue SCC | 2 |
| 50 | F | Mandible | 4a |
| 81 | F | Anterior 2/3 of Tongue | 4 |
| ? | ? | Buccal Mucosa | 2 |
| 59 | M | Right Mandibular SCC | 4a |
| 49 | M | Anterior floor of mouth | 3 |
| 57 | F | Right Neck Dissection | 0 |
| | | Tumour Node | |
| 71 | F | Left Floor Mouth | 4 |
| 73 | M | Floor of Mouth | 4 |
| 77 | F | Left Buccal | 1 |
| 62 | M | Right Floor Mouth | 1 |
| 60 | M | Right Floor Mouth | 4a |
| 49 | M | Left Floor Mouth | 3 |
| 85 | F | Right Buccal | 2 |
| 35 | F | Tongue | 2 |
| 63 | M | Tongue | 4 |
| 77 | F | Left Inferior Retromolar | 4a |
| | | Trigone | |
| 61 | M | Left Inferior Alveolus SCC | 4a |
| 71 | F | Soft Palate | 4 |
| 53 | ? | Right Tongue | 2 |
| 78 | M | Left Tongue | 3 |
| 82 | F | Tonsil | 1 |
| 75 | F | Left Tonsillar | 1 |
| 72 | F | Base Of Left Tongue | 3 |
| 50 | M | Base of Tongue | 4 |
| 78 | M | Tonsil | 2 |
| 82 | F | Total Laryngectomy | 2 |
| 87 | M | Right Glottic SCC | 4 |
| 96 | M | Glottis | 1 |
| 64 | M | Pharyngeal (Recurrent SCC) | 4a |
| 55 | M | Right Hypopharynx | 4 |
| 62 | M | Non tumour control | |
| 39 | M | Non tumour control | |
| | M | Non tumour control | |
| 32 | M | Non tumour control | |
| 54 | M | Non tumour control | |
| 71 | F | Non tumour control | |
| 66 | M | Non tumour control | |
| 61 | F | Non tumour control | |
| 63 | M | Non tumour control | |
| 51 | F | Non tumour control | |
| 58 | F | Non tumour control | |

Serum was also collected from 11 healthy (non-HNSCC) subjects (average age 55.7 years; 63% males) to be used as controls in this study (see also Table 3 above).

From each subject, 5 mL of blood was collected directly into BD Vacutainer® blood collection tubes. All samples were collected at room temperature (prior to surgery in the case of HNSCC sufferers) and serum was isolated by centrifuging BD Vacutainer® Tubes at 800 rpm for 10 mins. The serum-containing supernatant was collected and aliquoted into Eppendorf 1.7 ml tubes and stored at −80° C. for RNA isolation.

For total RNA extraction and purification serum stored at −80° C. was thawed on ice for approximately 15 minutes. 400 μl of serum was slowly dispensed into a freshly labelled RNase/DNase free Eppendorf tube and diluted with 100 μl of RNase free H₂O and proteinase K at a concentration of 1 mg/ml. This mixture was incubated at 37° C. for 20 minutes to elute any proteins. Tri-Reagent RT-LS (Molecular Research Centre) was added to the solution in an amount 1.5 times the volume of the mixture together with 100 μl of bromoanisole to homogenise. The homogenate was inverted for 5 seconds and decanted into a labelled 2 ml phase lock tube. This was centrifuged at 12000 g for 20 minutes at 4° C. Following this, at least 1 ml of the resulting aqueous solution was decanted into a fresh DNA Eppendorf Lobind tube. 5 μl of glycogen at 5 mg/ml and 500 μl of 100% isopropanol were added to the aqueous solution, mixed by inversion, and incubated overnight at −20° C. The sample was then centrifuged for 20 minutes at 12000 g at 4° C. The clear supernatant was discarded and the remaining pellet flash spun for 2 minutes at 16000 g. The clear solution surrounding the pellet was removed and the pellet washed twice with 1 mL cold 70% ethanol by centrifugation at 10000 g for 10 minutes. The pellet was resuspended in 10 μl RNase free H₂O. The re-suspended RNA was quantitated using a Nanodrop UV-Vis spectrophotometer and the RNA quality assessed using an Agilent 2100 Bioanalyser.

Total RNA samples pooled into four groups: non-tumour control; oral cavity tumour; oropharyngeal tumour; and pharyngeal/laryngeal tumour. For each group total RNA was assessed using a Nanodrop 1000 spectrophotometer (Thermo Scientific). After purification, the quality and integrity of the total RNA was determined using an Agilent 2100 Bioanalyzer (Agilent Technologies, Inc.) and the percentage of small RNAs (predominantly miRNAs) determined. Each of the four pooled total RNA samples contained small RNAs ranging in size between 10 and 40 nucleotides (data not shown). The samples with the most prominent small RNA population were the pooled oral cavity tumour and pooled pharyngeal/laryngeal tumour samples (76% and 93% small RNA/miRNA, respectively). The oropharyngeal tumour pooled sample contained 54% small RNA/miRNA, and the non-tumour control pooled sample contained 49% small RNA/miRNA.

Pooled total RNA samples were then subjected to miRNA expression analysis using an Agilent 8×60K miRNA microarray platform (Agilent Technologies, Inc.). These microarrays enable highly sensitive detection of miRNAs, across a dynamic range of more than 5 logs, with a comprehensive coverage of miRNAs found in the miRBase database (www.mirbase.org) The differential expression of miRNAs between each of the HNSCC subgroups (oral cavity, oropharyngeal and pharyngeal/laryngeal) and the non-tumour control group was then determined using Partek Genomic Suite 6.6 Beta. Differential miRNA expression was also determined between the HNSCC samples (as a single group) and the non-tumour control group.

Differential expression patterns were analysed using volcano plots. Cut-offs of 2-fold changes (up or down) and p-values with FDR of less than 0.05 were used in the initial analyses. After compilation of initial lists, miRNAs were selected with p-values of less than 0.000001.

To limit variability and increase robustness amongst array data and RT-qPCR quantification values, data was exported as described hereafter. Microarray miRNA analysis during the discovery stage was analysed using Partek Genomic Suite (Partek, USA) with results verified in an early-stage validation cohort using the pre-amplification RT-qPCR technique according to the manufacturer's instructions (Applied Biosystems). All late-stage validation RT-qPCR raw fluorescence analysis files were exported from the Step One plus in an EXCEL spreadsheet formatted for LinRegPCR analysis. Baselines were determined and amplicon groups were assigned with the following exclusion criteria: samples under analysis; samples without amplification; samples without plateau phase; samples with low Cq value (>37); and samples being outside of the 5% of the group median efficiency per amplicon. Furthermore, a log linear phase parameter during estimation of baseline was included. Quantitative PCR efficiencies were exported and statistically analysed. All samples were processed by this optimised LinRegPCR protocol, accurately assessing the endogenous abundance of each amplicon within every sample analysed. The abundance of amplicon in the samples analysed was demonstrated without being confounded with a reference gene. Once these values were considered accurate, a threshold of true and false values was determined statistically using both Cq (Cq<37) and NO values. These normalized datasets were then analysed, and differential miRNA expression levels between individuals with oral squamous cell carcinoma and healthy individuals in the early- and late-stage validation recorded. Seven candidate overexpressed miRNAs were chosen from the array according to the criteria of stringent array analysis of over-expressed miRNAs with significant p-values and greater than ten times fold change between the data set of healthy individuals and those with oral squamous cell carcinoma.

To construct the miRNA diagnostic classifier, thereby differentiating individuals with oral squamous cell carcinoma from healthy controls, the Cq data obtained from RT-qPCR was applied to the following classification models: Logistical Regression; Logistical Regression with k-fold validation; Machine learning; and Machine learning with k-fold validation. The ideal miRNA diagnostic classifier, denoted herein as Tri_{miR} was established with Logistical Regression modelling.

The inventors also carried out Cox Proportional Hazard modelling in the late-stage validation group investigating the association between the survival time of patients monitored over 4 years and eight predictor variables; Age, Sex and abundance of six miRNAs.

### Example 1

### Differential miRNA Expression Between HNSCC and Non-Tumour Control Groups

The inventors first investigated the expression of miRNAs in the compiled pooled HNSCC serum samples (n=52) compared to the non-tumour control serum samples (n=11). Volcano plot analysis identified 53 miRNAs significantly over expressed (up regulated) in sera from HNSCC subjects compared to sera from non-tumour control subjects, and 40 miRNAs the expression of which was significantly under expressed (down regulated).

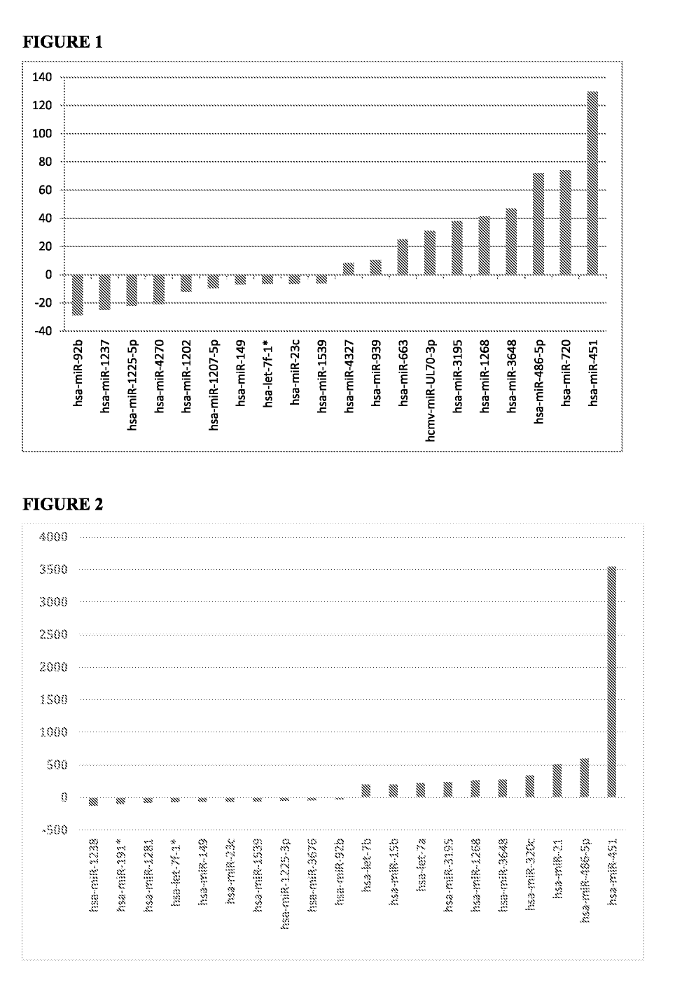

FIG. 1 shows the ten most over expressed and ten most under expressed miRNAs with p-values less than 0.000001 in sera from HNSCC subjects compared to sera from non-tumour control subjects, as determined from the volcano plot analysis. Table 4 presents the p-value and fold change of six of the most over expressed and three of the most under expressed miRNAs from FIG. 1, together with the number of known gene targets of each miRNA.

TABLE 4

| | | | |
|---|---|---|---|
| microRNA | p-value | Fold change | Gene Targets |

Over expressed miRNAs

| | | | |
|---|---|---|---|
| miR 451 | 1.32E−09 | 129.732 | 14 |
| miR-720 | 2.97E−07 | 73.9955 | 5 |
| miR-486-5p | 1.42E−08 | 72.0939 | 106 |
| miR-1268 | 1.09E−10 | 41.2989 | 9 |
| miR-939 | 1.64E−08 | 10.3577 | 97 |
| miR-4327 | 1.08E−10 | 8.15604 | — |

Down regulated miRNAs

| | | | |
|---|---|---|---|
| miR-1202 | 3.58E−10 | −12.0132 | 179 |
| miR-1237 | 1.28E−07 | −24.989 | 177 |
| miR-4270 | 3.08E−08 | −20.7995 | — |

### Example 2

### Differential miRNA Expression Between Oral Cavity Tumour Group and Non-Tumour Control Group

The inventors then investigated the expression of miRNAs in the oral cavity tumour pooled serum samples (n=42) and the non-tumour control samples (n=11). Volcano plot analysis identified 130 miRNAs significantly over expressed (up regulated) in sera from subjects with oral cavity tumours compared to sera from non-tumour control subjects, and 36 miRNAs the expression of which was significantly under expressed (down regulated).

FIG. 2 shows the ten most over expressed and ten most under expressed miRNAs with p-values less than 0.000001 in sera from subjects with oral cavity tumours compared to sera from non-tumour control subjects, as determined from volcano plot analysis. The majority of differentially expressed miRNA were found to be over expressed (between 100 to 1000 fold increased). The most under expressed miRNA, miR-129-3p, exhibited a relatively modest reduction in expression of 100 fold.

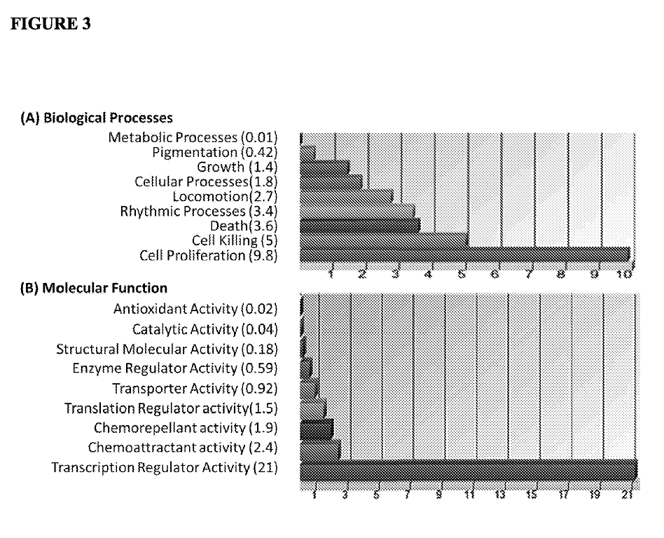

Gene ontology mapping of the differentially expressed miRNAs in oral cavity tumour samples indicated a high enrichment score for transcriptional regulator activity and cell proliferation (FIG. 3). The predicted gene targets involved in transcriptional regulation and cell proliferation of four of the most over expressed miRNAs in FIG. 2 are summarised in Table 5. One of these miRNA, miR-21, was not found to be differentially expressed in serum from subjects having either oropharyngeal or pharyngeal/laryngeal tumours (see Examples 3 and 4 below), suggesting the potential applicability of this miRNA as a single biomarker for oral cavity squamous cell carcinomas. It is noted that miR-21 has in excess of 200 putative gene targets.

TABLE 5

| | |
|---|---|
| miRNA | Gene targets |

Cell proliferation

| | |
|---|---|
| miR-486-5p | PIM1, PTEN |
| miR-21 | CDC25A, PTCH1, TGFB1, YAP1, ZFP36L2 |
| miR-320c | BCAT1, E2F1, ENPEP, EVI5, KITLG, MYH10, PTEN |
| Let-7a | BCAT1, CBFA2T3, CDC25A, CDV3, FGF5, MXD1, |
| | OSMR, TNFSF9, TP53, TUSC2, UHRF1, UHRF2 |

Transcriptional regulation

| | |
|---|---|
| miR-486-5p | ARID1A, FOXO1, MKL1, NKX2-3, SMAD2, TBL1X |
| miR-21 | ALX1, CL2, ELF2, GLIS2, PPARA, YAP1 |
| milk-320c | CDKN1C, CNOT7, E2F1, E2F3, ESRRG, FOXO3, |
| | FOXq1, GABPB2, HDAC4, HLTF, IKZF2, MYST4, |
| | MYT1L, NR3C1, ONECUT1, PGR, SP1, TBL1XR1, ZIC3 |
| Let-7a | ANKRD49, CD86, COL1A1, ELF4, FOXP1, GABPA, |
| | HAND1, HOXA9, IKZF2, MEF2D, PPARA, PPARGC1A, |
| | SMAD2, TEAD3, TP53, WNT1, NPAT, TEAD1 |

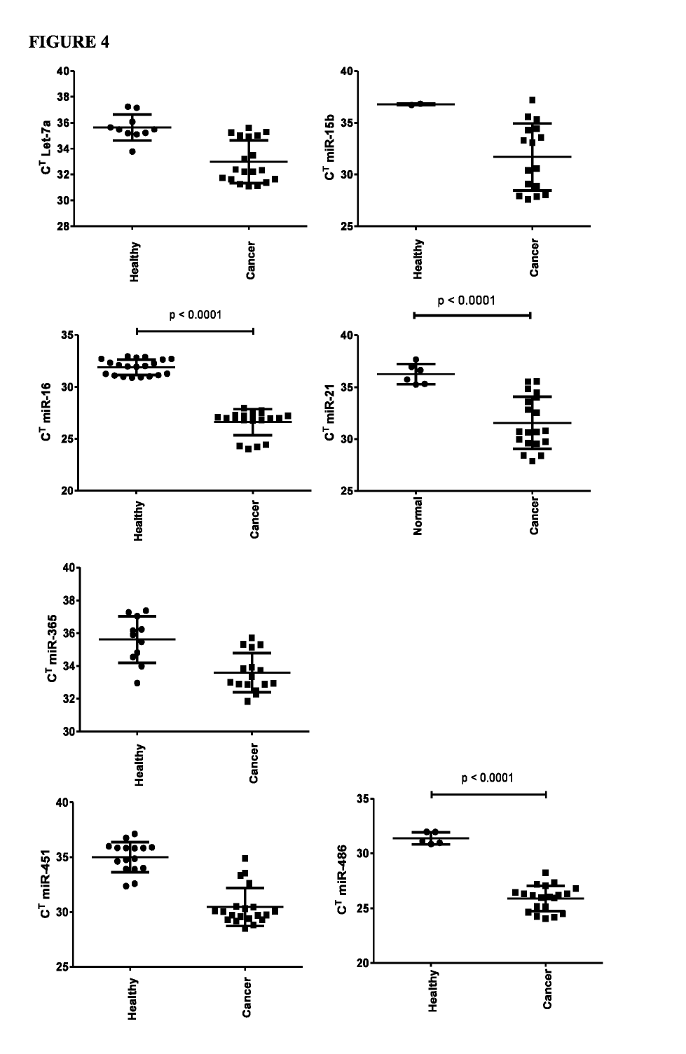

Further analysis of the microarray data revealed a cohort of seven miRNAs showing strong predictive power in diagnosing oral squamous cell carcinomas, each having a p-value of less than 0.000001: let-7a, miR-15b, miR-16, miR-21, miR-365, miR-451 and miR-486. One of these, miR-16 did not appear as one of the ten most over expressed miRNAs in the oral cavity tumour samples (see FIG. 2). The inventors confirmed the expression of these seven miRNAs in patient samples by qPCR using TagMan® Gene Expression Assays (Applied Biosystems). The TaqMan® protocol was modified, employing Applied Biosystems TagMan® Master Mix (2×)-2.50 μL, TagMan® Gene Probe (20×)-0.5 μL, Diluted cDNA-1.0 μL, RNAse-free Deionized Water-1.0 μL. The small volume qPCR reaction increases the sensitivity for detecting specific miRNAs. All reactions performed in triplicate were conducted using either the StepOnePlus™ or the QuantStudio 12K Flex system (Life Technologies, USA). The data was then analysed using ΔCT or ΔACT. The PCR thermal parameters were 95° C. for 10 minutes, followed by 40 cycles at 95° C. for 15 seconds and extension at 60° C. for 60 seconds. The results (FIG. 4) clearly demonstrate that these miRNAs are elevated only in the oral cancer sera when compared to healthy sera.

### Example 3

### Differential miRNA Expression Between Oropharyngeal Tumour Group and Non-Tumour Control Group

The inventors also investigated the expression of miRNAs in the oropharyngeal tumour pooled serum samples (n=5) and the non-tumour control samples (n=11). Volcano plot analysis identified 19 miRNAs significantly over expressed (up regulated) in sera from subjects with oroparyngeal tumours compared to sera from non-tumour control subjects, and 15 miRNAs the expression of which was significantly under expressed (down regulated).

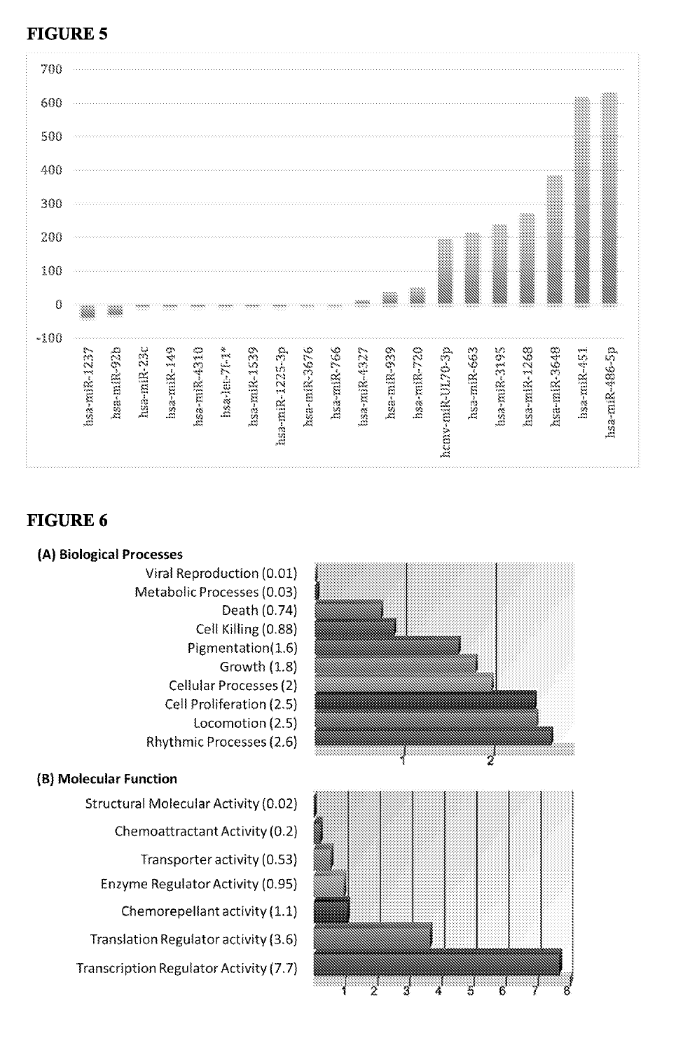

FIG. 5 shows the most over expressed and most under expressed miRNAs with p-values less than 0.000001 in sera from subjects with oropharyngeal tumours compared to sera from non-tumour control subjects, as determined from volcano plot analysis. As with the oral cavity tumour sample data (Example 2), the majority of differentially expressed miRNA were found to be over expressed (between 100 to 1000 fold increased).

The most over expressed miRNA was miR-486-5p (388-fold change). As noted above in Example 2, miR-486-5p was also highly expressed in serum from subjects with oral cavity tumours (72-fold increase). In contrast, miR-129* the highest down regulated miRNA was 51 fold lower in the tumour serum.

From a gene ontology analysis, the main ontology groups associated with the differentially expressed miRNAs, were, in terms of biological processes, rhythmic processes, locomotion and cell proliferation, and in terms of molecular function was transcription regulator activity (FIG. 6). This latter functional group is a particular focus of the miRNA identified as being the most over expressed in oropharyngeal serum samples, miR-486-5p (see FIG. 4), with over 100 known gene targets. This data and the finding of significant overexpression of the same miRNA in oral cavity tumour samples suggests the possibility of a central role for miR-486-5p in HNSCC. It is also noted that one of the miRNAs identified as being significantly over expressed in serum from subjects having oropharyngeal tumours (also significantly over expressed in serum from subjects having oral cavity tumours; Example 2) has a very large number of putative target genes (in excess of 530).

### Example 4

### Differential miRNA Expression Between Pharyngeal/Laryngeal Tumour Group and Non-Tumour Control Group

The inventors also investigated the expression of miRNAs in the pharyngeal/laryngeal tumour pooled serum samples (n=5) and the non-tumour control samples (n=11). Volcano plot analysis identified 8 miRNAs significantly over expressed (up regulated) in sera from subjects with oroparyngeal tumours compared to sera from non-tumour control subjects, and 14 miRNAs the expression of which was significantly under expressed (down regulated).

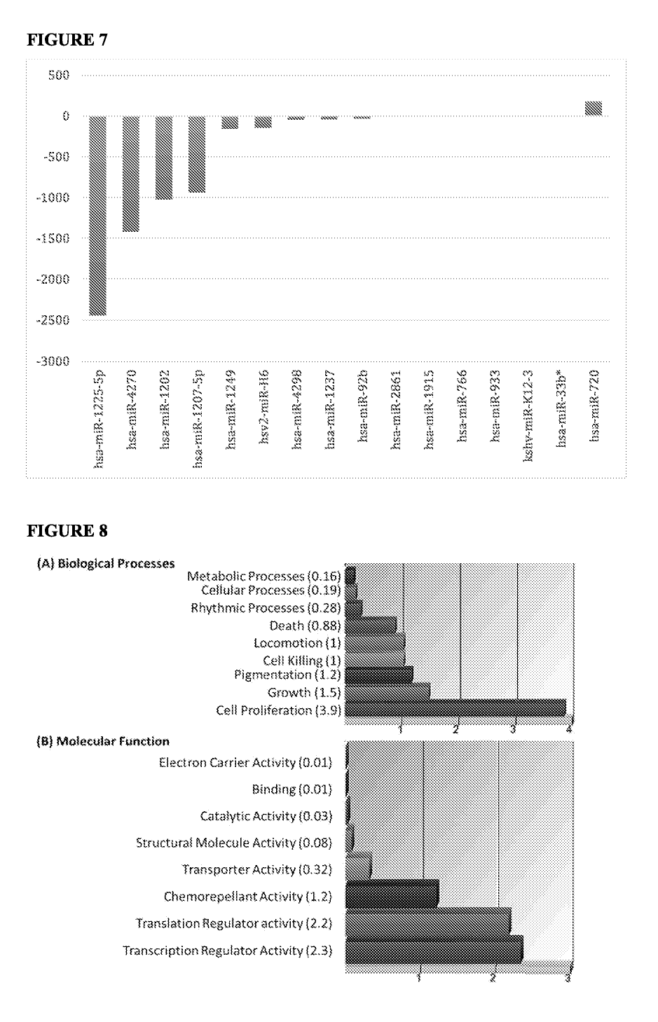

FIG. 7 shows the most over expressed and most under expressed miRNAs with p-values less than 0.000001 in sera from subjects with pharyngeal/laryngeal tumours compared to sera from non-tumour control subjects, as determined from volcano plot analysis. Immediately apparent from this data is the greater fold change observed in the under expressed miRNAs when compared to the over expressed miRNAs. For example expression miR-1225-5p was found to be reduced by 800 fold in serum from subjects with pharyngeal/laryngeal tumours, compared to non-tumour controls, whereas miR-720 (the most over expressed miRNA) was over expressed by only 80 fold. This pattern of differential serum miRNA expression was unique to the pharyngeal/laryngeal group, as the oral and oropharyngeal tumour samples showed a general over expression of miRNAs.

From a gene ontology analysis, the main ontology groups associated with the differentially expressed miRNAs were cell proliferation and transcription regulator activity (FIG. 8). The differentially expressed miRNAs identified as regulating the most targets within these ontology groups were miR-92b, miR-1225-5p, miR-1202, miR-1207-5p, miR-630 and miR-129-3p (all significantly under expressed in serum from subjects with pharyngeal/laryngeal tumours).

It is noted that miR-129-3p and miR-92b were found to be significantly under expressed in all three HNSCC tumour groups compared to non-tumour controls.

### Example 5

### Hemolysis is Not a Factor in Serum miRNA Expression Profiles

In miRNA analysis from serum, a common concern is the possibility of hemolysis in the sample and the effect this may have on the miRNA population. Using the Drabkin Assay as a measure of free haemoglobin, and qPCR, the inventors have demonstrated that the serum samples from subjects with oral cavity tumours were not hemolysed. To test the impact of haemolysis, serum samples were subjected to physical disruption by multiple freeze thaw cycles, boiling and vigorous mixing. This involved pipetting the serum over a period of five minutes at room temperature to ensure complete hemolysis of the cells contained within the serum.

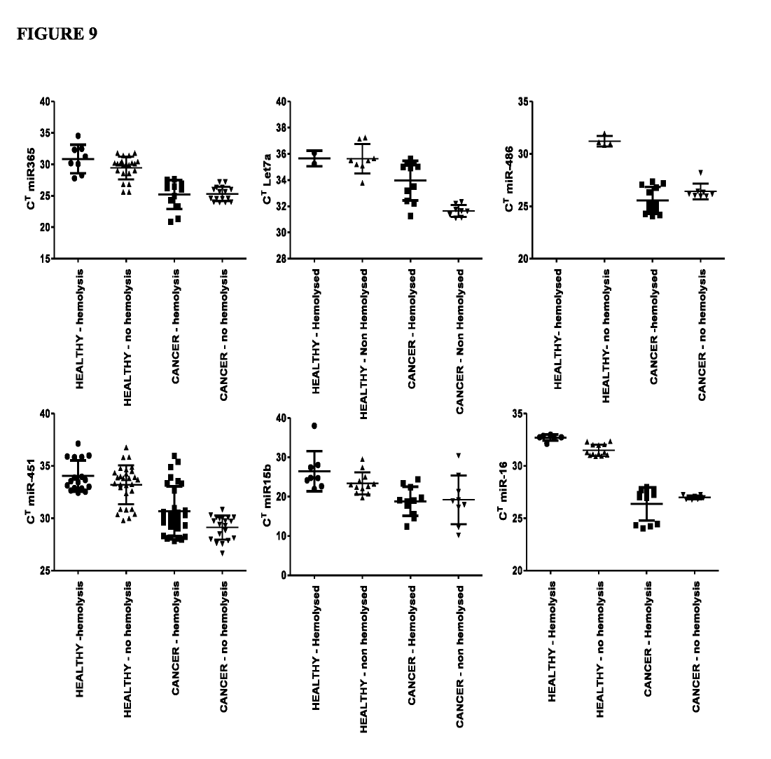

As shown in FIG. 9, the differential expression of six miRNA (miR-365, let-7a, miR-486, miR-451, miR-15b and miR-16) between oral cavity tumour serum samples and non-tumour control serum samples was not affected by hemolysis. The data in FIG. 9 also suggest the potential of each of these miRNAs as biomarkers for the diagnosis of oral squamous cell carcinoma.

### Example 6

### Validation of miRNA Expression and of Diagnostic and Prognostic Value of miRNA Signatures

Six miRNAs (let-7a, miR-16, miR-21, miR-451, miR-486-5p and miR-92a-3p) identified as being upregulated in head and neck cancers were validated using an alternative assay. This alternative assay, referred to as RNAmp, is an adaption of the TaqMan methodology.

Serum samples from 76 patients with head and neck cancer (of varying subtypes) and serum samples from healthy patients were obtained. Total RNA was extracted as described above and resuspended in a 100 μl RNase free dH₂O before RNA quality and concentration was assessed with the Nanodrop 1000 (Thermoscientific) and Qubit Fluormeter 2.0 (Thermofisher). The isolated RNA was then formulated at concentrations of 5 ng/μl, 10 ng/μl, 15 ng/μl or 30 ng/μl for use in first strand cDNA synthesis, and assessed individually.

First strand cDNA synthesis was performed using the High-capacity TaqMan® miRNA Reverse Transcription Kit. Briefly, to generate the 15.0 μl miRNA synthesis cDNA reaction, 4 U of 20 U/μL RNase inhibitor, a total volume of 6 μl miRNA RT primer mix, 50 U of MultiScribe™ Reverse Transcriptase, 50 Units/μL 1× volume of 1.0×RT Buffer, and 1 mM dNTP were combined and mixed with between 10 and 50 ng total RNA.

The cDNA product was diluted 1:4 and 1 μL was added to a RT-PCR master mix containing 0.5 μL 20× TaqMan® Assay, 5 μL 2× TaqMan® Universal PCR Master Mix, an 3 μL water (final volume of 10 μL). Triplicate qRT-PCR reactions were then carried out using an Applied Biosystems Step One machine with the following thermal-cycling procedure: 95° C. for 10 min, followed by 40 cycles of 95° C. for 15 s and 60° C. for 1 min (1.6° C./s ramp rate) as specified by the manufacturer (Applied Biosystems). The data was then analysed using ΔCt.

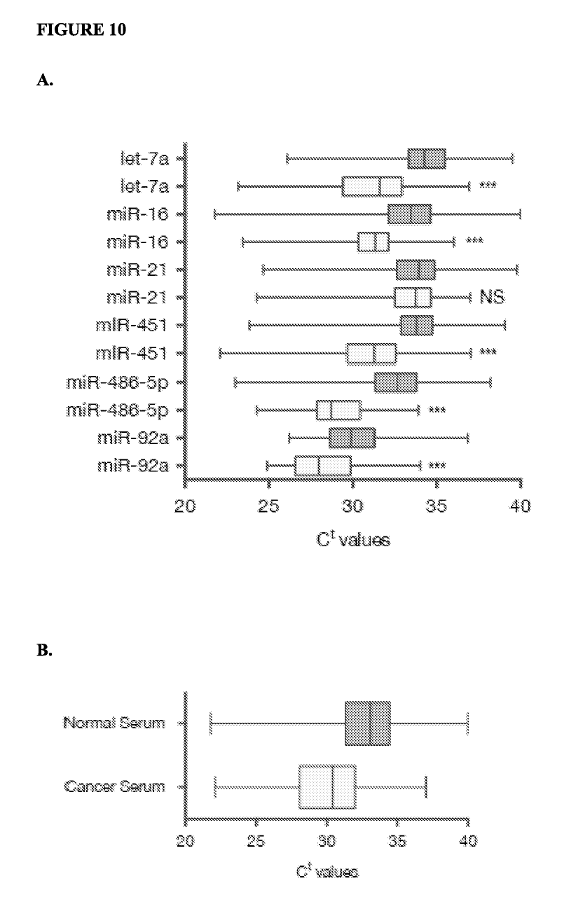

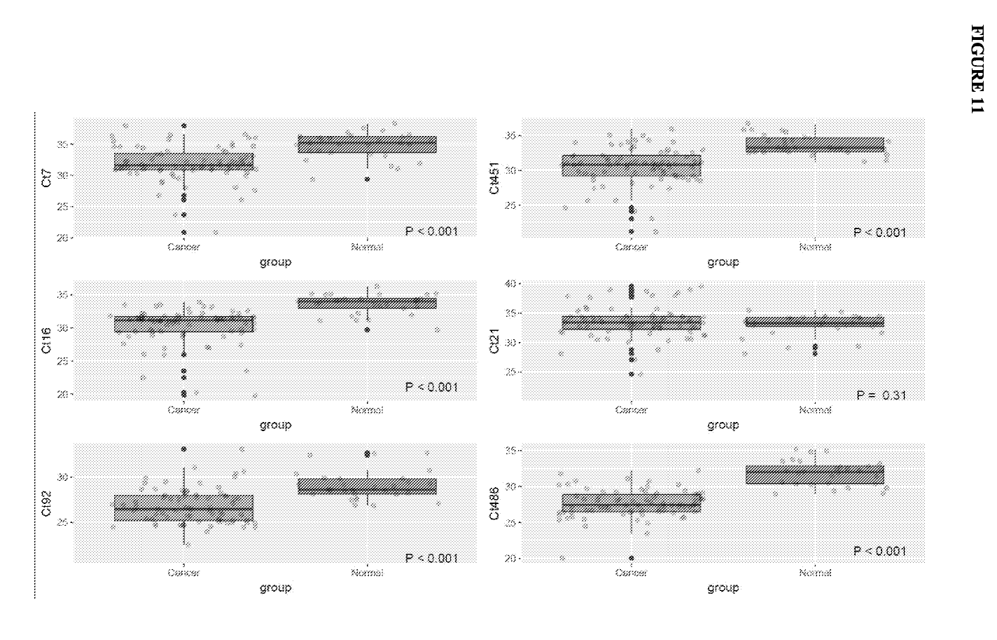

The results (FIG. 10A) clearly show that let-7a, miR-16, miR-451, miR-486-5p and miR-92a-3p are elevated in sera from patients with head and neck cancer when compared to sera from healthy patients. In this large cohort study, the expression of miR-21 in sera from patients with cancer was only slightly higher when compared to expression in sera from healthy. The difference between this observation and the data shown in FIG. 2 for oral cancer may be due to the fact that the patients in this cohort presented with a range of head and neck cancer subtypes (not just oral cancer), which may skew the data.

The average of these six miRNAs in combination is elevated in patients with head and neck cancer in comparison to healthy patients, and can thus be used to discriminate patients with head and neck cancer from healthy patients (FIG. 10B).

An overview of the dataset quality was developed by plotting data inter-correlation between the six aforementioned miRNAs. The distribution was found to be consistent across the markers. The analysis demonstrated the strong diagnostic discriminatory power of the markers, in particular miR-486-5p and miR-92a-3p [0.84] (data not shown).

A box plot (R-Studio) categorisation of Cq values for the suite of six miRNAs (let-7a, miR-16, miR-451, miR-486-5p and miR-92a-3p) across oral squamous cell carcinoma samples and healthy controls is shown in FIG. 11). The cancer group exhibited very small variation in their values across all miRNAs. Hsa-miR-486-5p and hsa-miR-92a-3p embodied the clearest difference, with no overlapping in the 75th percentiles. From this clear separation, it is possible to distinguish between cancerous samples and normal (non-cancerous) samples.

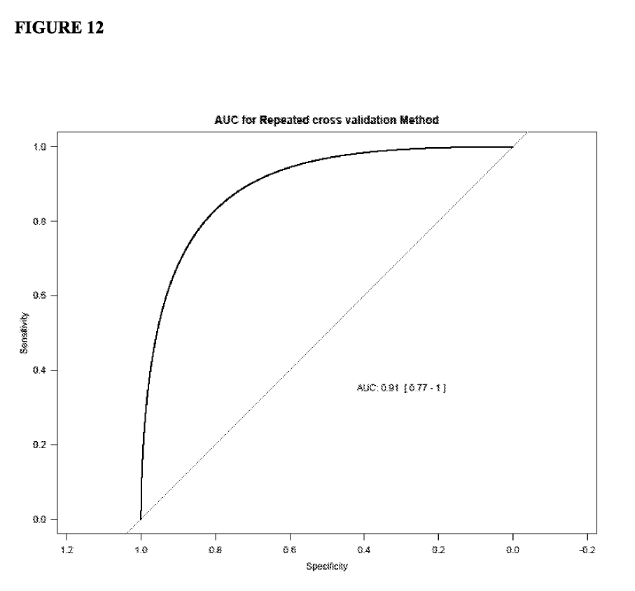

The inventors then determined the efficacy of the diagnostic signature using single or multiple miRNA markers selected from the aforementioned miRNAs. The clinical criterion correlated to pathology findings of positive or negative oral squamous cell carcinoma diagnosis. To construct the miRNA diagnostic classifier, thereby differentiating individuals with oral squamous cell carcinoma from healthy controls, the Cq data obtained from RT-qPCR was applied to the following classification models: logistical regression; logistical regression with k-fold validation, machine learning, and machine learning with k-fold validation. A specific miRNA diagnostic signature classifier, denoted herein as Tri_{miR}, was established with logistical regression modelling with an AUC 0.9 [0.734-0.978], a sensitivity of 91.3 and specificity of 85.7 (FIG. 12). The Tri_{miR} signature constitutes the three miRNAs hsa-miR-16, hsa-miR-92a-3p and hsa-miR-486-5p. (The signature comprising all six miRNAs-let-7a, miR-16, miR-21, miR-451, miR-486-5p and miR-92a-3p—is herein referred to as ‘6_{miR}’.) The accuracy of the final Tri_{miR} diagnostic and 6_{miR} survival score occurred in two validation sets, early and late. The predicted probability of being diagnosed as any stage of oral squamous cell carcinoma by Tri_{miR} was calculated according to the following formula (in which the miRNA designator is substituted with the calculated Cq value for that miRNA). Those skilled in the art will appreciate that this formula is exemplary only of the formulae that may be employed.

Logit [p=OC] wherein Log p/1−p=(−)59.5+0.73×hsa-miR-16+(−)2.23×hsa-miR-92-3p+3.27×hsa-miR-486-5p.

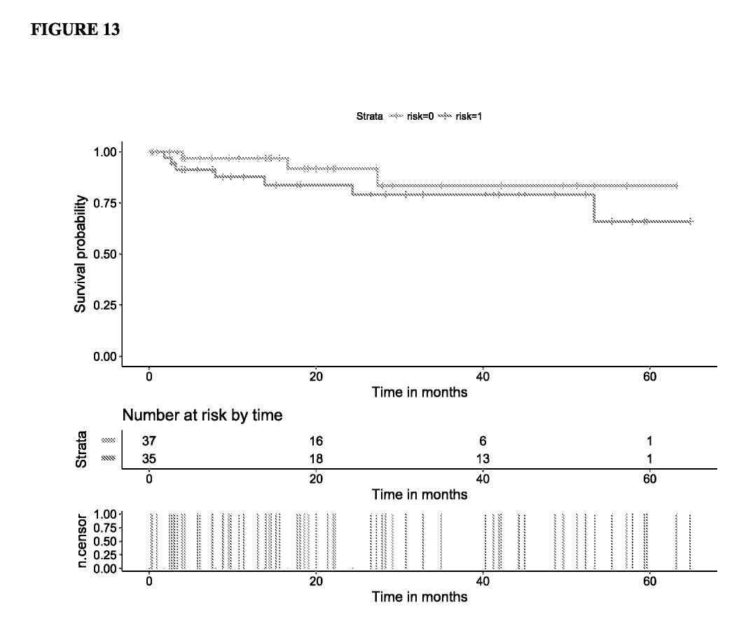

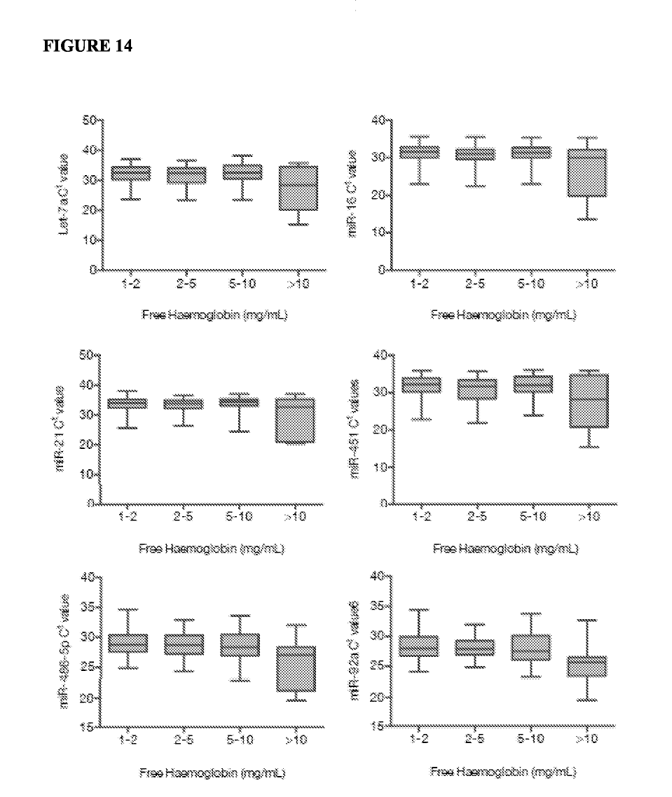

The polygenic 6_{miR} signature was associated with a low survival probability upon diagnosis. A personalised linear score ranked an individual having oral squamous cell carcinoma with a risk of survival, alongside a high score (4.8) of a 6_{miR} signature placing them at a 3.1 fold increase in dying within 4 years (FIG. 13). Using the following formula (in which the miRNA designator is substituted with the calculated Cq value for that miRNA), a risk score of over 4.8 indicated a higher chance of death upon initial diagnosis:

let-7a×(−0.4729)+hsa-miR-451×0.5305+hsa-miR-16×0.2646+hsa-miR-21×(−0.2593)+hsa-miR-92a-3p×(−0.6423)+hsa-miR-486-5p×0.4272.

### Example 7

### Validation of miRNA Expression Under Haemolytic Conditions

The effect of haemolysis on expression of let-7a, miR-16, miR-21, miR-451, miR-486-5p and miR-92a-3p was also assessed using the RNAmp assay. Haemolysis was induced as described above in Example 5 in serum samples from patients with head and neck cancer and free haemoglobin levels were assessed. The samples were then classified into 4 subcategories of haemolysis (1-2 mg/mL free haemoglobin; 2.5 mg/mL free haemoglobin; 5-10 mg/mL free haemoglobin; and >10 mg/mL free haemoglobin) before miRNA expression levels were assessed using the RNAmp assay as described in Example 6.

As shown in FIG. 11, expression of each of the six miRNAs was only compromised under conditions of severe haemolysis (>10 mg/mL free haemoglobin). All six miRNAs exhibited consistent CT values throughout the three lower grades of haemolysis (1-2 mg/mL free haemoglobin; 2.5 mg/mL free haemoglobin; and 5-10 mg/mL free haemoglobin).

Two confounding factors for the molecular analysis of all samples, hemolysis and concentration of total RNA input, were assessed with a quality control platform comprising gold standard hemolysis testing of all sera, and stratified samples according to the degree of blood disruption. Two different RNA concentrations were scrutinised for their capacity to deliver consistent Cq values. Statistical testing ensued, with deviation between the medians of each of the cohorts in question measured using a Student t-test, a Welch t-test and Wilcoxon sum rank test. These analyses confirmed that the diagnostic and prognostic value of the miRNAs described herein are not compromised under haemolytic conditions, nor are Cq values adversely impacted by varying RNA concentration (data not shown).

### Example 8

### Stability of Serum miRNA

To determine if collection and storage conditions of blood samples affects the detectable expression of miRNA biomarkers, blood samples were collected and stored at either room temperature or 4° C. before serum levels of the miRNAs were tested. Specifically, blood was drawn from healthy volunteers and then serum was isolated from the blood at room temperature. Samples were then either stored at 4° C. or room temperature and processed every 24 hours for a duration of 7 days. Expression of let-7a, miR-16, miR-21, miR-451, miR-486-5p and miR-92a-3p was assessed using the RNAmp assay as described above.

It was observed that there was no significant difference in the Ct values of the six miRNAs when compared across the two storage temperatures (data not shown). This suggests that the biomarkers are stable for up to seven days at either room temperature or at 4° C.

1. A method for detecting a head and neck cancer of the oral cavity or throat in a subject, the method comprising executing the step of determining the expression of two or more miRNA in a biological sample obtained from a subject, wherein the two or more miRNA are selected from the group consisting of hsa-let-7a, hsa-miR-16, hsa-miR-21, hsa-miR-451, hsa-miR-486-5p and hsa-miR-92a-3p, and wherein the level of expression of said two or more miRNAs in the biological sample relative to the level of expression of said two or more miRNAs in one or more cancer-free reference samples is indicative of the presence of a head and neck cancer of the oral cavity or throat of the subject.

2. A method according to claim 1, wherein the head and neck cancer of the oral cavity or throat is an oral, oropharyngeal, pharyngeal or laryngeal cancer.

3. A method according to claim 1 or 2, wherein the cancer is a squamous cell carcinoma.

4. A method according to any one of claims 1 to 3, wherein the biological sample obtained from the subject is a blood sample.

5. A method according to claim 4, wherein the blood sample is a whole blood or blood serum sample.

6. A method according to any one of claims 1 to 5, comprising t determining the expression of three or more, four or more, five or more of six or more of said miRNAs.

7. A method according to claim 6, wherein the method comprises determining the expression of hsa-let-7a, hsa-miR-16, hsa-miR-21, hsa-miR-451, hsa-miR-486-5p and hsa-miR-92a-3p.

8. A method according to claim 6, wherein the method comprises determining the expression of hsa-miR-16, hsa-miR-486-5p and hsa-miR-92a-3p.

9. A method for detecting a head and neck cancer of the oral cavity or throat in a subject, the method comprising executing the step of determining the expression of the miRNAs hsa-miR-16, hsa-miR-486-5p and hsa-miR-92a-3p in a biological sample obtained from a subject, wherein an increase in the level of expression of said miRNAs in the biological sample relative to the level of expression of said miRNAs in one or more cancer-free reference samples is indicative of the presence of oral cancer in the subject.

10. A method according to claim 9, wherein the cancer is oral squamous cell carcinoma.

11. A method for predicting the probability of survival of a subject having a head and neck cancer of the oral cavity or throat, the method comprising executing the step of determining the expression of the miRNAs hsa-let-7a, hsa-miR-16, hsa-miR-21, hsa-miR-451, hsa-miR-486-5p and hsa-miR-92a-3p in a biological sample obtained from a subject having oral cancer, wherein an increase in the level of expression of said miRNAs in the biological sample relative to the level of expression of said miRNAs in one or more cancer-free reference samples is indicative of a reduced likelihood of survival of the individual beyond about four years.

12. A method according to claim 11, wherein the cancer is oral squamous cell carcinoma.

13. A kit for use in screening for head and neck cancers of the oral cavity and throat, wherein the kit comprises one or more reagents for determining the expression of one or more miRNAs as defined in any one of claims 1 to 12.

14. A computer system or apparatus, configured to aid in the detection or diagnosis of a head and neck cancer of the oral cavity or throat, wherein computer software is employed to analyse data relating to the expression of one or more miRNAs as defined in any one of claims 1 to 12, in a biological sample obtained from a subject and to provide a diagnostic prediction with respect to the subject.

15. A computer system or apparatus according to claim 14, wherein the computer software is also employed to compare said data to data relating to the expression of the one or more miRNAs in one or more cancer-free reference samples.

16. A method for selecting a subject for treatment for a head and neck cancer of the oral cavity or throat, the method comprising:
(a) executing a step of determining the level of expression of one or more miRNAs as defined in any one of claims 1 to 12 in a biological sample obtained from a subject, wherein the level of expression of the at least one miRNA in the biological sample relative to the level of expression of the at least one miRNA in one or more cancer-free reference samples is indicative of the presence of a head and neck cancer of the oral cavity or throat of the subject; and
(b) selecting a subject, identified in (a) as having a head and neck cancer of the oral cavity or throat, for treatment for said cancer.

17. A protocol for monitoring the efficacy of a therapeutic treatment for a head and neck cancer of the oral cavity or throat, the protocol comprising:
(a) obtaining from a subject a first biological sample, wherein the first biological sample is obtained before or after commencement of treatment;
(b) obtaining from the same subject a second biological sample, wherein the second biological sample is obtained at a time point after commencement of treatment and after the first biological sample is obtained;
(c) executing the step of measuring for expression of at least one miRNA in the first and second biological samples, wherein the at least one miRNA is as defined in any one of claims 1 to 12; and
(d) comparing the expression of the at least one miRNA in the first biological sample with the expression of the same at least one miRNA in the second biological sample;
wherein a change in the expression of the at least one miRNA between the first and second biological samples is indicative of whether or not the therapeutic treatment is effective.

18. A protocol according to claim 17, further comprising obtaining and executing steps in respect of a third or subsequent sample.

19. A protocol according to claim 17 or 18, for use in the screening of candidate agents for treating the cancer.


SEQUENCE LISTING (44 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
uagcagcacaucaugguuuaca

SEQ ID NO: 4 - RNA, 22 nt, Homo sapiens
uagcagcacguaaauauuggcg

SEQ ID NO: 5 - RNA, 22 nt, Homo sapiens
uagcuuaucagacugauguuga

SEQ ID NO: 6 - RNA, 20 nt, Homo sapiens
gugcauugcuguugcauugc

SEQ ID NO: 7 - RNA, 20 nt, Homo sapiens
ggggaugggcuggcgcgcgg

SEQ ID NO: 8 - RNA, 20 nt, Homo sapiens
aaaagcuggguugagagggu

SEQ ID NO: 9 - RNA, 23 nt, Homo sapiens
agggacuuuugggggcagaugug

SEQ ID NO: 10 - RNA, 22 nt, Homo sapiens
aaaccguuaccauuacugaguu

SEQ ID NO: 11 - RNA, 22 nt, Homo sapiens
uccuguacugagcugccccgag

SEQ ID NO: 12 - RNA, 22 nt, Homo sapiens
aggcggggcgccgcgggaccgc

SEQ ID NO: 13 - RNA, 18 nt, Homo sapiens
accuccggggucgcucua

SEQ ID NO: 14 - RNA, 22 nt, Homo sapiens
aggaggaauuggugcuggucuu

SEQ ID NO: 15 - RNA, 22 nt, Homo sapiens
ugugcgcagggagaccucuccc

SEQ ID NO: 16 - RNA, 24 nt, Homo sapiens
uggggagcugaggcucugggggug

SEQ ID NO: 17 - RNA, 18 nt, Homo sapiens
cgggcguggugguggggg

SEQ ID NO: 18 - RNA, 22 nt, Homo sapiens
accuugccuugcugcccgggcc

SEQ ID NO: 19 - RNA, 19 nt, Homo sapiens
ggggccuggcggugggcgg

SEQ ID NO: 20 - RNA, 17 nt, Homo sapiens
cgcgccgggcccggguu

SEQ ID NO: 21 - RNA, 21 nt, Homo sapiens
agccgcggggaucgccgaggg

SEQ ID NO: 22 - RNA, 19 nt, Homo sapiens
ggcuugcaugggggacugg

SEQ ID NO: 23 - RNA, 23 nt, Kaposi′s sarcoma-associated herpes virus
ucacauucugaggacggcagcga

SEQ ID NO: 24 - RNA, 22 nt, Homo sapiens
ugagguaguagauuguauaguu

SEQ ID NO: 25 - RNA, 22 nt, Homo sapiens
aucacauugccagugauuaccc

SEQ ID NO: 26 - RNA, 22 nt, Homo sapiens
agggacgggacgcggugcagug

SEQ ID NO: 27 - RNA, 23 nt, Homo sapiens
ucuggcuccgugucuucacuccc

SEQ ID NO: 28 - RNA, 23 nt, Homo sapiens
caacggaaucccaaaagcagcug

SEQ ID NO: 29 - RNA, 22 nt, Homo sapiens
aggaggaauuggugcuggucuu

SEQ ID NO: 30 - RNA, 21 nt, Homo sapiens
gugccagcugcagugggggag

SEQ ID NO: 31 - RNA, 21 nt, Homo sapiens
uggcagggaggcugggagggg

SEQ ID NO: 32 - RNA, 22 nt, Homo sapiens
ugagccccugugccgcccccag

SEQ ID NO: 33 - RNA, 22 nt, Homo sapiens
guggguacggcccagugggggg

SEQ ID NO: 34 - RNA, 21 nt, Homo sapiens
cgggggcggggccgaagcgcg

SEQ ID NO: 35 - RNA, 23 nt, Homo sapiens
gugagugggagccccagugugug

SEQ ID NO: 36 - RNA, 24 nt, Homo sapiens
aggagggaggagaugggccaaguu

SEQ ID NO: 37 - RNA, 17 nt, Homo sapiens
ucgccuccuccucuccc

SEQ ID NO: 38 - RNA, 21 nt, Homo sapiens
uccugcgcgucccagaugccc

SEQ ID NO: 39 - RNA, 15 nt, Homo sapiens
aggagauccuggguu

SEQ ID NO: 40 - RNA, 20 nt, Homo sapiens
ucagggagucaggggagggc

SEQ ID NO: 41 - RNA, 22 nt, Homo sapiens
cugggacaggaggaggaggcag

SEQ ID NO: 42 - RNA, 16 nt, Homo sapiens
gcagcauucauguccc

SEQ ID NO: 43 - RNA, 20 nt, Herpes simplex virus-2
aauggaaggcgaggggaugc

SEQ ID NO: 44 - RNA, 22 nt, Homo sapiens
uauugcacuugucccggccugu